(19) United States
(12) Patent Application Publication
(10) Pub. No.: US 2023/0390461 A1
(43) Pub. Date: Dec. 7, 2023

(54) TISSUE SCAFFOLDS AND CONSTRUCTS

(71) Applicant: UNIVERSITY OF WOLLONGONG, New South Wales (AU)

(72) Inventors: Jeremy Crook, New South Wales (AU); Eva Crook-Tomaskovic, New South Wales (AU); Samuel Rath-Bone, New South Wales (AU)

(21) Appl. No.: 18/249,074

(22) PCT Filed: Oct. 19, 2021
(86) PCT No.: PCT/AU2021/051216

§ 371 Date: Apr. 14, 2023

(30) Foreign Application Priority Data
Oct. 19, 2020 (AU) 2020903779

(51) Int. Cl. A61L 27/52; C12N 5/00; A61L 27/56; A61L 27/44; A61L 27/38; A61N 1/36; A61N 1/378

(52) U.S. Cl. CPC A61L 27/52; C12N 5/0068; A61L 27/56; A61L 27/446; A61L 27/3834; A61L 27/3878; A61N 1/36125; A61N 1/3787; C12N 2513/00; C12N 2533/00; A61L 2430/32; A61L 2400/12; A61N 2007/0026

(57) ABSTRACT

An electrostimulatable 3-dimensional (3D) electrogel scaffold comprising piezoelectric nanoparticles uniformly dispersed throughout a homogenous hydrogel polymer matrix, wherein the hydrogel polymer matrix is gelled and comprises crosslinked alginate, carboxymethyl-chitosan and agarose polymers.

### FIELD OF THE INVENTION

The invention relates to tissue scaffolds, tissue engineering and tissue regeneration constructs and methods for preparing same.

### BACKGROUND

Human stem cells such as neural stem cells and induced pluripotent stem cells (iPSCs) have the ability to self-renew for large-scale expansion whilst maintaining the capacity to differentiate to other cell types of the human body. These qualities, together with the potential for autologous application, make iPSCs compelling candidates for cell replacement therapies, tissue and organ engineering, and pharmacology and toxicology screening.

Since their discovery a decade ago, the development of culture protocols for human iPSCs has primarily focused on clinical compliance, cell line stability, and efficiency of differentiation to desired cell lineages, all the while retaining conventional monolayer (2D) culture. However, 2D methods of cell culture are not representative of actual cell environments within tissues and organs of the human body. Therefore, cells cultured as monolayers on flat surfaces and isolated from physiologically relevant inputs, as such, are intrinsically poorly predictive in vivo behaviour, form and function, limiting their value for basic research through to translation and use including pharmaceuticals development.

Recent interest in recapitulating the 3D cytoarchitecture of native tissues in vitro to better simulate cell behavior in vivo, together with advances in fabricating bioactive, mechanically tunable and biocompatible materials are driving the application of 3D configured biomaterials for stem cell research and therapy. For example, by mimicking important features of a target tissue including the extracellular microenvironment, 3D-biomaterials have the potential to instruct cell fate and function in ways not previously attainable. The small number of 3D systems for iPSC culture reported to date rely on the ability of iPSCs to self-organize when seeded onto or cast within supporting material such as conventional tumor-derived Matrigel basement membrane or more defined polymeric scaffolds.

An alternative approach to bioengineering 3D iPSC constructs involves advanced 3D bioprinting for direct-write (or co-) printing of stem cells together with biomaterial to reproducibly generate tissue of a desired architecture. Co-printing represents a single-step approach to rapidly fabricate a 3D cellularized construct whereby iPSCs are immediately integrated with biomaterials by encapsulation for direct and complete contact with extracellular elements that more closely mimic the native cell microenvironment.

Prior art methods involving nanoparticles typically endeavor to wrap the nanoparticles with a polymer (e.g., poly-L-lysine, polyethylene imine, glycol chitosan) coating to enhance the nanoparticle biocompatibility as well as to facilitate internalization of the nanoparticles into cells of interest. Polymer wrapping of nanoparticles is considered essential in the art to provide homogenous nanoparticle dispersions that are aggregate-free and which are easily internalized in the target cells. For example, WO2010/119403 teaches electrical stimulation of cells with internalized PLL wrapped BNNTs (non-covalent wrapping of the nanotubes with PLL) in cell media. This document teaches that the level of internalization of the piezoelectric BNNTs influxes the effectiveness of the cell stimulation. Dispersions in PBS were achieved by ultrasonication of BNNT power in a 0.1% PLL solution in PBS for 12 hours at a sonicator output of 20 W.

### SUMMARY OF THE INVENTION

In a first aspect the invention provides a 3-dimensional (3D) electrogel scaffold comprising piezoelectric nanoparticles uniformly dispersed throughout a homogenous hydrogel polymer matrix, wherein the hydrogel polymer matrix is gelled and comprises crosslinked alginate, carboxymethyl-chitosan and agarose polymers.

In a related embodiment, there is provided a 3-dimensional (3D) electrogel scaffold comprising piezoelectric nanoparticles uniformly dispersed throughout a porous homogenous hydrogel polymer matrix, wherein the porous hydrogel polymer matrix is gelled and comprises crosslinked alginate, carboxymethyl-chitosan and agarose polymers. Preferably, on leaching of a portion of the carboxymethyl-chitosan out of the 3D electrogel scaffold, the 3D electrogel scaffold takes the form of a porous hydrogel polymer matrix comprising interconnected pores that form channels or pathways throughout the scaffold that support ingress, invasion and infiltration of cells, oxygen and nutrients throughout the scaffold or construct.

In a second aspect the invention provides a three-dimensional (3D) electrogel precursor solution for forming a 3D electrogel scaffold according to the first aspect, the precursor comprising:

an aqueous hydrogel polymer solution comprising a homogeneous mixture of dissolved alginate, dissolved carboxymethyl-chitosan and dissolved agarose polymers, and piezoelectric nanoparticles uniformly dispersed throughout the aqueous hydrogel polymer solution.

In a third aspect the invention provides a method of forming a 3D electrogel scaffold comprising the steps of:
(i) providing an 3D electrogel precursor solution according to the second aspect;
(ii) forming the 3D electrogel precursor solution into a desired 3D shape or 3D pattern;
(iii) crosslinking the hydrogel polymers of the 3D shape or 3D pattern to form a 3D electrogel scaffold having the desired 3D shape or 3D pattern, wherein the 3D electrogel scaffold comprises a uniform dispersion of piezoelectric nanoparticles throughout a gelled and crosslinked hydrogel polymer matrix, wherein the hydrogel polymer matrix comprises a homogenous mixture of alginate, carboxymethyl-chitosan and agarose polymers.

In a related embodiment, the method comprises the additional step of: allowing a portion of the carboxymethyl-chitosan to leach out of the 3D electrogel scaffold thereby forming a porous hydrogel polymer matrix comprising interconnected pores that forms channels or pathways throughout the scaffold that support ingress, invasion and infiltration of cells, oxygen and nutrients throughout the scaffold or construct.

In a fourth aspect the invention provides a 3D electrogel scaffold obtained by the method of the second aspect. In a related embodiment, the 3D electrogel scaffold comprises encapsulated cells to form a cell laden 3D electrogel scaffold. On culturing in cell culture media or in vivo in a suitably supporting cell proliferation environment, the cell laden 3D electrogel scaffold results in the formation of a 3D electrogel tissue engineered construct. On electrical stimulation by ultrasound-mediated piezoelectric stimulation (USPZ) of the nanoparticles in the construct, the 3D electrogel tissue engineered construct is converted into an advanced 3D electrogel tissue engineered construct.

In a fifth aspect the invention provides a use of a 3D electrogel scaffold according to the first aspect or a 3D electrogel scaffold or a 3D electrogel tissue engineered construct or an advanced 3D electrogel tissue engineered construct of the fourth aspect in a medical application.

In a sixth aspect the invention provides a use of a 3D electrogel scaffold according to the first aspect or a 3D electrogel scaffold or a 3D electrogel tissue engineered construct or an advanced 3D electrogel tissue engineered construct of the fourth aspect in the repair and/or regeneration of tissue malfunction or injury, in vitro or in vivo.

In a seventh aspect the invention provides an electric nerve guide comprising a support and a 3D electrogel scaffold according to the first aspect or a 3D electrogel scaffold or a 3D electrogel tissue engineered construct or an advanced 3D electrogel tissue engineered construct of the fourth aspect disposed on said support, wherein the support is adapted to encase injured nerves.

In an eight aspect the invention provides a use of an electric nerve guide according to the seventh aspect in the repair and/or regeneration of tissue injury or tissue dysfunction, such as nerve injury, peripheral nerve injury or peripheral nerve regeneration.

In a ninth aspect the invention provides a method of repairing and/or regenerating a tissue malfunction or injury comprising providing the 3D electrogel scaffold according to the first aspect or a 3D electrogel scaffold or a 3D electrogel tissue engineered construct or an advanced 3D electrogel tissue engineered construct of the fourth aspect in the form of an implant to an area of tissue or organ injury or tissue or organ dysfunction.

In a tenth aspect the invention provides a method of repair and/or regeneration of tissue malfunction or injury comprising the steps of:
providing a 3D electrogel scaffold, a 3D electrogel tissue engineered construct or an advanced 3D electrogel tissue engineered construct as described herein as an implant;
positioning the implant at the site of the malfunctioned tissue or injured tissue;
electrically stimulating the implant by ultrasound-mediated piezoelectric stimulation (USPZ) to promote repair and/or regeneration of tissue malfunction or injury at the implant site.

Preferably, the implant is in the form of a nerve guide including a 3D electrogel component as described herein.

### BRIEF DESCRIPTION OF THE DRAWINGS

FIG. 1 illustrates the results of frequency sweep measurements of 3D electrogel precursor solution with varying concentrations of BTNPs compared to control gel sol (without BTNPs). Ratio of storage (G′) and loss (G″) modulus taken as tan δ;

FIG. 2 illustrates the results of strain sweep measurement of 3D electrogel precursor solution with varying concentrations of BTNPs compared to control gel sol (without BTNPs). Ratio of storage (G′) and loss (G″) modulus taken as tan delta;

FIG. 3 illustrates the results of flow curve measurement of the shear thinning 3D electrogel precursor solution with varying concentrations of BTNPs compared to control gel (without BTNPs), showing dynamic viscosity (shear stress/shear rate) of 3D electrogel precursor solution;

FIG. 4 illustrates a 3D printed electrogel scaffold;

FIG. 5 illustrates live (Calcein AM) and dead (propidium iodide (PI)) cell staining of hNSC-laden electrogel. (A, B) Lower and higher magnification images respectively of live (top row) and dead cells (bottom row; single cells and cell aggregates) within electrogel. (C, D) High magnification fluorescent and overlaid bright field (BF) images, respectively of live cells within electrogel, with incorporated nanoparticles also apparent as dark particles amongst cell aggregates. (E, F) 3D reconstruction of hNSC-laden-electrogel, showing both cells and BTNPs with extensive neurite extensions emanating from hNSCs;

FIG. 6 illustrates live (Calcein AM; top row) and dead (propidium iodide (PI); bottom row) cell staining of hNSC-laden control (no BTNPs) gel. A, B and C) Lower, and higher magnification images respectively of live and dead cells within control gel;

FIG. 7 illustrates immunocytochemical analysis of differentiated hNSCs within: (A) control gel (without BTNPs) and (B) electrogel (with BTNPs), showing early neuronal cell marker TUJ1 (middle column) and glial cell marker GFAP (middle column) Cell nuclei were stained with blue-fluorescent DNA stain DAPI (4′,6-diamidino-2-phenylindole; left column) and regions with neurites (insets) are highlighted by arrow heads;

FIG. 8 illustrates immunocytochemical analysis of printed hNSC-laden electrogel constructs following days differentiation with or without 7 days concurrent USPZ stimulation. (A) USPZ stimulation resulted in significant and complex neuritogenesis, evident as many neurite extensions, including axons with varicosities (A-inset; arrow heads), projecting from mature MAP2 (microtubule-associated protein 2) expressing neurons over long distances to synapse with other neurons and associated GFAP+neuroglia (˜200 um in length; arrow heads). (B) Stimulated constructs exhibited greater numbers of discrete, large, compact and uniformly distributed clusters/aggregates of neural cells, with more neurites and bundles of neurites radiating per cluster (B-inset) compared to (C) unstimulated electrogel constructs. (D) Compared to unstimulated electrogel controls (Electrogel—US), stimulated (Electrogel+US) neurites exhibited increased numbers of synaptic varicosities/boutons (swellings) formed along their length (0.106±0.022 vs 0.192±0.025 respectively, Mean±SEM; n=8; *P<0.05; Welch's t-test with normal distribution confirmed by a D'Agostino-Pearson test). Although stimulated neurites also exhibited higher mean numbers of spines (protrusions) compared to unstimulated controls, the difference was not statistically significant (0.106+/−0.023 vs 0.064+/−0.019 respectively, Mean±SEM.; n=8; P=0.183; Welch's t-test with normal distribution confirmed by a D'Agostino-Pearson test). (E) In the absence of ultrasound, the ratio of Tuj1 vs GFAP labelling revealed an increased neuronal relative to glial cell induction within electrogel compared to control gel (without BTNPs; 1.328±0.044 vs 0.454±0.044 respectively, Mean±SEM; n=12; *P<0.001), which was further enhanced by USPZ stimulation, being greater than unstimulated (no ultrasound exposure) electrogel constructs (1.606±0.037 vs 1.328±0.044 respectively, Mean±SEM.;**P<0.05; n=12). Notably, there was no effect of ultrasound exposure on the ratio of neuronal Tuj1 labelling vs glial cell GFAP labelling for control gels (0.454±0.044 [−US] vs 0.429±0.052 [+US], Mean±SEM.; P=0.704; n=12). Statistical analysis of the ratios of Tuj1 vs GFAP labelling was performed using two-way ANOVA with Holm-Sidak's post-hoc test (F(1,8)=11.70, P=0.009), with Spearman's Homogeneity of Variance Test and D'Agostino-Pearson test for normal distribution, to confirm the statistical assumptions for ANOVA were satisfied. For all images, cell nuclei are stained with blue-fluorescent DNA stain DAPI. Statistical analyses were performed in GraphPad Prism8;

FIG. 9 illustrates live-cell calcium imaging of printed hNSC-laden electrogel constructs following 10 days differentiation with or without 7 days concurrent USPZ stimulation. USPZ stimulated cells exhibited an increased spontaneous firing rate (peaks per minute) compared to unstimulated cells. Mean±SEM (n=3). Student t-test. P<0.05;

FIG. 10 illustrates live-cell calcium imaging of printed hNSC-laden electrogel constructs following 10 days differentiation with or without 7 days concurrent USPZ stimulation. USPZ stimulated cells exhibited an increased spontaneous spike amplitude (average peak amplitude) compared to unstimulated cells. Mean±SEM (n=4). Student t-test. P<0.05;

FIG. 11 illustrates peripheral nerve repair technology. (A) Integrated multi-modal technology depicting injured and repaired nerve and electric nerve-guide (e-nerve-guide; with inner printed electro-gel and outer protective collagen membrane) for (B) wireless ultrasound-mediated e-stim and nerve regeneration. Panel (B) depicts ulnar nerve repair of the forearm;

FIG. 12 illustrates electrocompacted type 1 collagen for outer membrane of the e-nerve-guide. A) Schematic of electro-compaction (EC) of collagen, whereby collagen molecules are repelled by both electrodes and compacted at the isoelectric point, due to the charges generated by the electrolysis of water. (B) Schematic and SEM images illustrating the relative alignment of collagen molecules and fibrils in conventional (non-EC; left panels) compared to EC (right panels) collagen. (C) Representative examples of EC verses non-EC collagen membranes. While both membranes can be handled with tweezers, EC membranes retain their shape due to greater alignment of collagen fibrils. (Figure taken from Chen et al, Acta Biomaterialia, Volume 113, 1 Sep. 2020, Pages 360-371).

FIG. 13 illustrates wireless e-stim augments nerve cell generation from native human neural stem cells, with increased neuronal networking and function for enhanced 3D neural tissue engineering. (A) Quantitative immunocytochemistry (Integrated Density; IntDen) of mature nerve cell marker MAP2 (left graph) and synaptic vesicle protein SYP (middle graph), and live-cell calcium flux imaging (right graph) indicating e-stim augments nerve cell generation, function and networking for enhanced 3D neural tissue formation. Two-way ANOVA with Bonferroni post hoc, ***p<0.001, n=3, Error bars: SEM. (B) E-stim promotes MAP2/SYP (left column/right column labelling; left panels) expressing nerve cell development from stem cells and functional linkage of networked cells (ie. functional connection [white lines; right panels] between firing neurons [white dots; right panels]).

### DETAILED DESCRIPTION OF THE INVENTION

Biomimetic cell cultures which better represent human cell growth and tissue outside the human body require new platforms that integrate biologically relevant human cell lines with advanced techniques for 3D tissue engineering and 3D electrostimulation. Herein the inventors describe a 3-dimensional (3D) electrogel scaffold, a 3D electrogel tissue engineered construct derived from a 3D electrogel scaffold, and an advanced 3D electrogel tissue engineered construct derived from the 3D electrogel tissue engineered construct, each of which comprise piezoelectric nanoparticles dispersed uniformly or homogenously through the entirety of the scaffold or construct material. It is believed that this is the first report of a 3-dimensional (3D) electrogel scaffold, a 3D electrogel tissue engineered construct derived from a 3D electrogel scaffold, and an advanced 3D electrogel tissue engineered construct derived from the 3D electrogel tissue engineered construct, which may be electrically stimulated by ultrasound-mediated piezoelectric stimulation (USPZ). The scaffolds and constructs of the invention arise from a scalable and versatile platform which may be employed for the electrogenic development of functional engineered tissue in vitro and in vivo, including organ tissue. The scaffolds and constructs of the invention can be used in applications including stem cell research, cell replacement therapies, tissue and organ engineering, pharmacology, toxicology screening, pharmaceutical development and regenerative medicine, implants, etc. The scaffolds and constructs of the invention are amenable to tissue development and function studies, including understanding how microenvironmental features affect cell and tissue phenotypes.

The technological platform described herein is exemplified in one embodiment by a 3D electrogel neuronal tissue engineered construct derived from a 3D electrogel scaffold comprising hNSCs, as well as the advanced 3D electrogel neuronal tissue engineered construct derived from the 3D electrogel neuronal tissue engineered construct of the invention. In particular, the inventors have successfully generated these neuronal and non-neuronal tissues in vitro. However, other tissue types are possible, e.g., where human iPSCs are used and suitable differentiation conditions are applied, to result in, for example, cardiac or bone tissue.

Other desirable features of the 3D electrogel scaffold, the 3D electrogel tissue engineered construct and the advanced 3D electrogel tissue engineered construct of the invention relate to their superior functionality in terms of cell viability, proliferation and differentiation. Such desirable features include one or more of: (i) suitable porosity for diffusion of cells, cell media, oxygen and nutrients throughout the entire scaffold or construct framework, and (ii) correct mechanochemistry of component biomaterials to promote cell adhesion, survival, networking and function.

The 3D electrogel scaffold on its own without cells finds application in various medical applications, for example, as an electric nerve guide/cuff for peripheral nerve repair following injury, as an electric nerve (sacral, tibial) cuff for urinary incontinence, as an electric electrogel patch for urinary incontinence by bladder muscle stimulation, as electric nerve (vagal) cuff for pancreatic islet insulin secretion and attenuation of hyperglycemia, as a cardiac pacemaker for regulating the electrical conduction system and therefore beating of the heart, as an injectable electrogel for deep brain stimulation (DBS) for treatment of movement disorders, including Parkinson's disease, essential tremor and dystonia; and as an injectable electrogel for DBS for the treatment of drug resistant neuropsychiatric disorders; a bone structure for bone replacement or repair.

A preferred embodiment of the invention is based on the encapsulation of cells, preferably encapsulated stem cells, within the polymers of the 3D electrogel scaffold. Encapsulated cells in a cell laden 3D electrogel scaffold may be successfully subjected to 3D cell culture and later 3D differentiation in appropriate culture media to provide the 3D electrogel tissue engineered construct of the invention. Where desired, the 3D electrogel tissue engineered construct of the invention can be subjected to one or more rounds of electrical stimulation via ultrasound-mediated piezoelectric stimulation (USPZ) for a desired period of time to produce the developmentally advanced 3D electrogel tissue engineered construct of the invention. The advanced tissue construct is characterized by homogeneity of advanced tissue features which are observed throughout the entirety of the advanced 3D electrogel tissue engineered construct of the invention. It will be understood that in the constructs of the present invention, cell proliferation, differentiation and maturation is augmented and enhanced homogenously throughout the construct by homogenous electrical stimulation of the construct through ultrasound-mediated piezoelectric stimulation (USPZ) of the nanoparticles which are homogenously dispersed through the scaffold. Prior to electrical stimulation, the encapsulated cells are allowed to proliferate and differentiate through contact with appropriate cell culture media, before the resultant construct is subjected to one or more rounds of electrical stimulation as described to give rise to the advanced 3D electrogel tissue engineered construct of the invention. The platform thus is capable of providing tissue engineering constructs whereby human stem cell proliferation and differentiation can be augmented homogeneously across the construct to produce homogenously matured engineered tissues. The platform is scalable and versatile, is amenable both in vitro and in vivo to any desired tissue or organ types. As reported herein, the concept has been exemplified in the case of a neural construct which results from electro-assisted differentiation of human NSCs (“hNSCs”) encapsulated in a 3D electrogel. However, the platform is amenable to other cell types including cardiac, bone, skin, pancreatic islet beta-cells, bone osteoblasts, and cartilage chondrocytes, etc.

### Scaffold/Construct

As used herein, by ‘construct’ means a tissue engineered construct which is a scaffold as described herein but comprising cells which has undergone cell culture and/or cell differentiation in appropriate cell culture media. The term ‘scaffold’ refers to the 3D biomaterial without cells or before cells are added. A cell laden 3D electrogel scaffold is one where cells have been added but culture has not yet taken place.

In one aspect, the invention describes a 3D electrogel scaffold (with or without cells), a 3D electrogel tissue engineered construct, and/or an advanced 3D electrogel tissue engineered construct comprising piezoelectric nanoparticles which are uniformly dispersed or homogenously dispersed throughout the scaffold or construct material. The scaffolds and constructs of the invention may be readily electrostimulated uniformly or homogenously throughout the scaffold or construct via application of ultrasound which uniformly and homogenously electrically activates the piezoelectric nanoparticles within the scaffold and produces desirable effects at the cellular level that result in the advance tissue state of the advanced 3D electrogel tissue engineered construct of the invention. For example, on application of ultrasound, mechanical forces are generated by the vibrating piezoelectric nanoparticles in the polymer matrix which are translated into a positive electrical charge on the surface of the nanoparticles. Cells that directly abut the piezoelectric nanoparticles are directly stimulated by the generated electrical charges. For example, such cells may experience attenuated membrane potentials which can result in changes in, for example, Ca²⁺ flux, that produce potential in the cells or other changes at the cellular level due to effects connected with attenuated ionic conductivities. In the case where a 3D electrogel scaffold without cells is used, for example, as an implant or patch on tissue or an organ, on electrical stimulation, these desirable changes are initiated at the interfaces of such tissues or organs and the scaffold. Electrically stimulated cells produce cell factors which can influence other cells including native cells and can thereby facilitate cell proliferation and functionality. Furthermore, where the 3D electrogel scaffold material is ionically conductive, the scaffold material may act as an electrolyte whereby electronic/ionic effects are transmitted through the electrolyte to influence other cells which are more remote from the nanoparticles. More specifically, as demonstrated herein in one exemplary embodiment, the invention results in functionally advanced 3D neural tissue which is characterised (in comparison to non-electrogel 3D based tissues) by the development of mature neuronal morphology underpinned by significant and complex neuritogenesis presenting as large numbers of discrete, uniformly distributed (throughout the construct) clusters and aggregates of cells with extensive neurite extensions (axon and dendrites) which project over long distances (over 200 nm) from sites of origin to synapse with other cells including neurons and neuroglia. The stimulated electrogel based constructs of the invention also exhibit greater numbers of discrete, large, compact and uniformly distributed clusters/aggregates of neural cells with more neurites and bundles of neurites radiating per cluster. In summary, compared to non electrogel based 3D constructs, the stimulated 3D electrogel tissue engineered constructs of the invention demonstrate augmented neural cell growth, neuronal induction and neuritogenesis with enhanced neural networking and tissue formation.

In the case of electrogel based cardiac tissue constructs and bone tissue constructs, the invention enables stimulation of component cells, including stem cells and other precursor cells during differentiation to augment cardiomyocyte development and bone cell (including but not limited to osteoblasts and osteocytes) development, cardiac tissue formation and bone tissue formation, and tissue function.

The piezoelectric nanoparticles are provided in the 3D electrogel scaffolds, the 3D electrogel tissue engineered constructs and the advanced 3D electrogel tissue engineered constructs of the invention as discrete piezoelectric nanoparticles, small aggregates of nanoparticles and/or combinations of discrete nanoparticles and nanoparticle aggregates. By “small aggregates” it is meant, aggregates of a few (e.g., 2 to 6) individual nanoparticles to form aggregates of diameter from about 600 nm to about 100 μm, from about 400 nm to 200 μm, for example, where a single particle may have a diameter of about 100 nm-200 nm. Regardless of their form, the nanoparticles are uniformly homogenously distributed throughout the hydrogel polymer matrix of the 3D scaffold and 3D construct. By ‘homogenously distributed’, it is meant that the nanoparticles are uniformly or evenly dispersed evenly distributed are located throughout the entire scaffold or construct polymeric matrix material. Preferably, the nanoparticles are encapsulated within the polymer matrix. The polymer matrix, particularly on crosslinking, locks the nanoparticles in position in the matrix thereby stabilizing the nanoparticle dispersion through the crosslinked polymer. This desirable nanoparticle distribution is understood to facilitate the advanced tissue development features homogenously across the entire scaffold/construct materials of the invention.

Suitably, where the polymer matrix comprises two or more polymers, the polymer matrix presents as a homogenous polymer matrix. By ‘homogenous polymer matrix’, it is meant the individual polymers are completely dispersed together and there are no beads, clumps or pockets of an individual, non mixed polymer located in any one or more single regions, areas, or parts of the polymer matrix.

Suitably, the invention provides a 3-dimensional (3D) electrogel scaffold, a cell laden 3D electrogel scaffold, a 3D electrogel tissue engineered construct or an advanced 3D electrogel tissue engineered construct, which comprises piezoelectric nanoparticles uniformly dispersed throughout a homogenous hydrogel polymer matrix, wherein the hydrogel polymer matrix is gelled and comprises crosslinked alginate, carboxymethyl-chitosan and agarose polymers. In a related embodiment, there is provided a 3-dimensional (3D) electrogel scaffold comprising piezoelectric nanoparticles uniformly dispersed throughout a porous homogenous hydrogel polymer matrix, wherein the porous hydrogel polymer matrix is gelled and comprises crosslinked alginate, carboxymethyl-chitosan and agarose polymers. Preferably, on leaching of a portion of the carboxymethyl-chitosan out of the 3D electrogel scaffold, the 3D electrogel scaffold takes the form of a porous hydrogel polymer matrix comprising interconnected pores that form channels or pathways throughout the scaffold which support ingress, invasion and infiltration of cells, oxygen and nutrients throughout the scaffold or construct.

Preferably, the 3-dimensional (3D) electrogel scaffold, a cell laden 3D electrogel scaffold, a 3D electrogel tissue engineered construct or an advanced 3D electrogel tissue engineered construct comprises a plurality of pores, suitably internal and external pores. Desirably, the scaffold or construct is a porous 3-dimensional (3D) electrogel scaffold, a porous cell laden 3D electrogel scaffold, a porous 3D electrogel tissue engineered construct or a porous advanced 3D electrogel tissue engineered construct. Preferably, the hydrogel polymers of the 3D scaffold adopt a porous structure having a mixture of larger and smaller pores. The pores can form channels or pathways throughout the scaffold or construct. Such channels or pathways may be interconnected and may aid in transport of nutrients, cells, air/oxygen and various factors throughout scaffold/construct. An arrangement where adjacent pores are interconnected to each other is particularly preferred. The arrangement presents are an irregular arrangement or irregular network of interconnected porous area. The pores may be in a structured arrangement or an unstructured arrangement. Suitably, the scaffold has areas having an internal pore size of 100 μm or less, more preferably 50 μm or less. In some embodiments, the entire scaffold has an internal pore size of 100 μm or less, more preferably 50 μm or less. A porous structure comprising a combination of interconnected large and small pores is preferred. For example, in one embodiment, a porous structure having a combination of large and small pores sized from 25-50 μm and 10-20 μm respectively has found to provide desirable results. Desirably, the interconnected pores produce a morphology having channels or pathways throughout the polymer matrix which support ingress, invasion and infiltration of cells, oxygen and nutrients throughout the scaffold or construct. Suitably, the porous structure may have a spongy or trabecular porous structure or morphology. Desirably, the morphology is one which allows for ingress, invasion and infiltration of cells, oxygen and nutrients throughout the scaffold or construct. Desirably, the nanoparticles, that is, individual piezoelectric nanoparticles or agglomerates of nanoparticles, are uncoated except for the crosslinked homogenous hydrogel polymer matrix of alginate, carboxymethyl-chitosan and agarose which encapsulate the nanoparticles. In some embodiments, the nanoparticles preferably do not comprise a distinct layer or coating of dispersant and/or cationic polymer. Only the homogenously mixed hydrogel polymer matrix which comprises alginate, carboxymethyl-chitosan and agarose surround the nanoparticles. In some embodiment, the nanoparticles are not wrapped or coated in a polymer which is separate or different to the homogenous polymer matrix of alginate, carboxymethyl-chitosan and agarose. In some embodiments, the nanoparticles are not distinctly wrapped or distinctly coated in a polyelectrolyte, such as PLL, PDL, or PEI or a cationic polymer typically used to aid in dispersion of nanoparticles.

In an alternative embodiment, the piezoelectric nanoparticles may be distinctly coated in a polymer or polyelectrolyte prior to dispersal of the nanoparticles in the homogenous hydrogel polymer matrix of alginate, carboxymethyl-chitosan and agarose. Preferably, the distinct coating or wrap of polymer or polyelectrolyte is a distinct cationic polymer or a distinct anionic polymer. Anionic polymers are preferred where this embodiment is desired. The distinct polymer or distinct polyelectrolyte coating or wrap around the nanoparticles in this embodiment is preferably not carboxymethyl-chitosan. Desirably, the piezoelectric nanoparticles may be dispersed with a distinct coating or wrap of polymer or polyelectrolyte selected from one or more of agarose, poly-D-lysine, poly-D-ornithine, or gum arabic.

In a preferred embodiment, the scaffold or construct of the invention comprises a uniform dispersion or distribution of cells throughout the entirety of the polymer matrix, thereby providing a cell laden 3D electrogel scaffold. Thus, suitably, a preferred 3D electrogel construct may comprises a uniform dispersion of cells throughout all of the polymer matrix. Desirably, the scaffold or construct comprises a uniform dispersion of cells throughout the porous hydrogel polymer matrix. The cell dispersion may be in the form of dispersed individual cells, aggregates of cells or a combination of individual cells or aggregates of cells homogenously dispersed throughout the polymer matrix.

In terms of cell types, the inventors have previously found that the 3D hydrogel scaffold component of the invention has broad and non-specific cytocompatibility for the support of all cell states including self-renewing and proliferating stem, progenitor or precursor cells (i.e., involving symmetric or asymmetric cell division; including but not limited to neural stem/progenitor cells and pluripotent stem cells) and non-self-renewing differentiated cells exemplified by cells arising from the three germ line lineages—endoderm, ectoderm, and mesoderm. The ectoderm gives rise to the nervous system and the epidermis, among other tissues, mesoderm gives rise to muscle cells and connective tissue, and endoderm gives rise to the gut and many internal organs. As exemplified herein, the nanoparticles as described and used herein are not associated with cell toxicity or death and so the electrogel of the invention is also expected to exhibit the same broad and non-specific cytocompatibility. Thus, suitably, the cells may be one or more types of stem cells. Desirably, the cells may include one or more cells selected from the group consisting of: adult stem cells, pluripotent stem cells or induced pluripotent stem cells (iPSCs) for ensuing differentiation to lineage specific cells. Suitably, the cells include but are not limited to nerve cells, glial cells, bone cells, cardiac cells including pacemaker cells, skin cells, cartilage cells, and bone cells. Desirably, the stem cells may be one or more of adult stem cells and pluripotent stem cells. The dispersed cells may include neural stem cells, bone cells, cardiac stem cells, epidermal stem cells, etc. The concentration of cells present depends on cell type. For example, in one embodiment, the optimal concentrations for human neural stem cells has been determined to be 1×10⁶ and pluripotent stem cells has been determined to be 2-8×10⁸.

Suitably, the piezoelectric nanoparticles may be in the form of nanospheres, nanofibers, nanotubes, nanocubes, or combinations thereof. In some embodiments, the piezoelectric nanoparticles may be selected from the group consisting of piezoelectric barium titanate nanoparticles (BTNPs), boron-nitride nanoparticles, and poly(vinylidene fluoride) (PVDF) nanoparticles. Preferred piezoelectric nanoparticles are cytocompatible. Preferred piezoelectric nanoparticles have a high piezoelectric coefficient. Preferred piezoelectric nanoparticles are hydrophobic. Preferred piezoelectric nanoparticles are barium titanate nanoparticle, preferably, in a tetragonal phase. Other piezoelectric nanoparticles are envisaged.

Preferably, the average diameter of nanoparticle agglomerations present throughout the polymer matrix are from 400 nm to 200 μm. In preferred embodiment, the diameters are ≤2000 nm, more preferably ≤1500 nm, more preferably ≤1000 nm, more preferably ≤500 nm, more preferably ≤350 nm, more preferably ≤300 nm. In some embodiments, preferred nanoparticles have average diameter of 00 nm. Preferred piezoelectric nanoparticles have an average particle diameter of about 200 nm, as good dispersions can be readily achieved using nanoparticles of this size. In embodiments where cell internalisation of the nanoparticles is preferred, using nanoparticles of particles of 500 nm is favoured as smaller nanoparticles are easier to internalize in cells. However, it is believed that at diameters of 00 nm, the piezo properties of the nanoparticle may not be optimal and so these sizes are less preferred.

Suitably, the piezoelectric nanoparticles may be present in the electrogel construct at a concentration of up to 1 mg/ml, preferably up to 3 mg/ml, preferably up to 5 mg/ml, preferably up to 7.5 mg/ml, preferably up to 10 mg/ml. In some preferred embodiments, the piezoelectric nanoparticles may be present at concentrations of from 1 to 5 mg/ml of the construct as it is believed that cytotoxicity may be less of a risk at these concentrations.

Desirably, the hydrogel polymers of the electrogel scaffold or construct may be ionically crosslinked, for example, ionic crosslinked using, for example, one or more divalent cations. Suitable divalent cations include a divalent metal cation such as Ca²⁺, Mg²⁺, Sr²⁺ and Ba²⁺. However, Ca²⁺ is preferred for biocompatibility reasons.

Desirably, the polymers may be crosslinked by irrigating, soaking, submerging or washing a 3D pattern or 3D form of electrogel precursor solution with a suitable crosslinking solution, preferably a solution of divalent ions, such as Ca²⁺ ions, for example. The divalent cations may be provided in the form of a solution of a suitable salt in distilled water or phosphate buffered saline (PBS). PBS solutions may be preferred as they more closely resemble physiological conditions and so may be more conducive to cell viability. A preferred Ca²⁺ ion solution is a CaCl₂) solution. The CaCl₂) concentration is preferably in the range of from about 10 mM to 200 mM. However, in some embodiments, a 50 mM solution of CaCl₂) has been found to be particularly preferred in terms of resulting in a scaffold with desirable flexibility, desirably porosity and desirable ability to support cell growth. Suitably, the crosslinking step may progress for a time period of from about 2 minutes to about 30 minutes prior to washing the resultant scaffold to remove the crosslinking solution. In some embodiments, crosslinking with the divalent metal solution may progress for 5, 10 or 20 minutes. In embodiments using 50 nM CaCl₂), 15 minutes is desirable. In some embodiment, on crosslinking with divalent metal ions, the resultant scaffold is ionically conductive and when media is provided to the scaffold, the media becomes imbibed within pores of the scaffold and may serve an electrolyte which assist in ion transport through the material. Thus, preferred 3D electrogel scaffold and constructs include hydrogel polymer matrices which are electrically conductive, for example, as evidenced by well-defined electrochemical responses with clear oxidation and reduction peaks in cyclic voltammetry studies and an impedance measurement, for example of, 225×10⁻⁴Ω.

Desirably, the hydrogel matrix comprises hydrogel polymers. Polysaccharide based hydrogel polymers or combinations of polysaccharide based hydrogel polymers are preferred. Biocompatible polysaccharide-based polymers or combinations of polysaccharide based polymers are particularly preferred. Hydrogel polymers or combinations of hydrogel polymers that support cell viability and more preferably cell proliferation and differentiation within a 3D scaffold formed from the hydrogel polymers are particularly preferred. For example, good results have been achieved with combinations of agarose, carboxymethyl chitosan and alginate. Suitably, the hydrogel matrix may comprise, for example, a combination of alginate, carboxymethyl-chitosan and agarose polymers. Alginate and agarose provide structural support for the construct, as well as low toxicity, and cytocompatible gelation. Alginate enables gelation in the presence of cations after printing. A range of different alginates can be used at a range of different concentrations. Carboxymethyl chitosan is a water-soluble derivative of chitosan and is conducive to cell survival within the construct, particularly following printing. Preferably, the carboxymethyl chitosan may have a deacetylation degree of >90%. It is believed that carboxymethyl chitosan may sustain cell survival by influencing gel porosity and permeability, for example, to oxygen and nutrients. Moreover, as a derivative of chitosan, carboxymethylchitosan is deemed to have low to absent toxicity, no mutagenic effects, affects cellular expression of growth factors, and promotes cell adhesion, migration, and proliferation.

Preferably the agarose is a low temperature gelling agarose. Agarose which gels at a temperature of from 30° C. to 45° C. may be used. However, particularly preferred agarose components are those that gel at physiological temperatures, for example, of about 37° C. Agarose that at least partially gelate (and can be maintained at) physiological temperatures are desirable for embodiments where cells are included in the precursor solution as this supports printability of the ink while providing an environment conducive to cell survival. In some embodiments, the polymers gel strongly at around 4° C. It will be appreciated that for embodiment involving printing, it is desirably to maintain a provided 3D pattern or 3D form ahead of crosslinking. Accordingly, it is preferred that bioprinting occurs at temperature of around 15° C. and more preferably at around 10° C. It has been found that these temperatures favour cell survival while providing an acceptable degree of 3D form retention to a bioprinted structure or pattern formed from precursor solution prior to crosslinking, while not cooling cells to an adverse degree that negatively affects their survival.

It is believed that in the case of a crosslinked scaffold, over time, at least a portion of the agarose polymer may leach out of the scaffold, e.g., over a period of about a week or so, leaving behind a porous structure comprising a combination of larger and smaller pores which are interconnected to each other. The resultant interconnected pores form channels or pathways throughout the polymer matrix that allows cells and media to invade and infiltrate through the scaffold. Furthermore, agarose has been found to provide the requisite viscosity for optimal bioprinting prior to gelation. Other known properties include high moisture retention of carboxymethyl chitosan, and antimicrobial and low inflammatory responses of both Al and carboxymethyl chitosan, all features conducive to cell support and survival.

Preferably, the alginate is sodium alginate. Suitably, the alginate is derived from seaweed. The alginate may be a low molecular weight alginate or a high molecular weight alginate. Low molecular weight alginates of 30 to 180 kDa are preferred. It is possible to disperse nanoparticles using low molecular weight alginate or a high molecular weight alginate. However, in embodiments involving cells, it is preferred to use a low molecular weight alginate as it is believed that low molecular weight alginate may be more desirable in terms of supporting cell growth. In a particularly preferred embodiment, the low molecular weight alginate has a molecular weight of around 50 kDa. Preferably, the alginate exhibits low viscosity of about 100 to 500 cP at 1% w/v concentration of alginate. In one embodiment, a preferred alginate has a viscosity of ≤300 cP at 1% w/v. In some embodiments, alginate with an M/G ratio of about 0.5 to about 2.0 may be used. However, preferred alginates have a M/G ratio of about 1.6. In some embodiments, alginate with an M/G ratio of about 1.6 to about 1.67 provides a particularly good crosslinked scaffold in terms of one or more of flexibility and durability particularly during electrical stimulation where ultrasound is applied to the scaffold. Less durable scaffolds are likely to break apart on application of ultrasound.

In a preferred 3D electrogel scaffold or 3D electrogel tissue engineered construct derived therefrom, the hydrogel polymer matrix may comprise a homogenous mixture of alginate, carboxymethyl-chitosan and agarose polymers. Desirably, these polymers may be provided in a ratio of 0.5 to 7.5:4 to 7:0.5 to 2.5 (% w/v), more preferably a ratio of 0.5 to 5:4.5 to 5.5:1.0 to 2.0 (% w/v), most preferably a ratio of 5:5:1.5 (% w/v).

The amount of alginate included depends on the optimal gel modulus required for a specific type of cell and the type of alginate, with the latter dependant on differences in the mannuronic acid (M) and guluronic acid (G) ratio (M/G) and block configuration (M blocks [M-M bonding], G blocks [G-G bonding] and M and G random blocks [M-G random bonding]).

In particular, a ratio of 5:5:1.5 (% w/v) alginate: carboxymethyl-chitosan: agarose polymers may provide an optimally viscous bioink that is conducive to cell survival during extrusion via an extrusion means such as syringe or a bioprinting head. In particular, suitable extrusion means include a syringe tip or an extrusion printhead for example. Further, this ratio of components may provide an extruded or printed bioink that maintains its printed 3D shape for a sufficient period of time to allow gelation to be induced, for example, up to 2 minutes at 10-20° C. In addition, it has been found that a bioink having a ratio of 5:5:1.5 (% w/v) alginate: carboxymethyl-chitosan: agarose polymers, when crosslinked, supports safe cell encapsulation, structural support, and sustained cell survival for the life of the construct.

Preferably, the 3D electrogel construct or scaffold arises from a casting, extrusion, injection or bioprinting process involving a suitable electrogel precursor solution or sol (described herein as a bioink for embodiments where extrusion or bioprinting is desired), which on gelation forms a mechanically robust biomaterial capable of encapsulating cells, and supporting their viability, proliferation and differentiation. It will be understood that a selected casting, extrusion, injection or bioprinting process provides the scaffold or construct with a desired shape or pattern, which on crosslinking forms a mechanically robust scaffold or construct.

Suitably, the 3D bioprinting involves extrusion printing. 3D bioprinting enables excellent control over the 3D architecture of a construct, including the spatial assembly of cells and materials for optimal tissue development. 3D bioprinted scaffolds and constructs and involve extrusion bioprinting of a suitable precursor solution, sol or bioink which is optimized for bioprinting. Such sols are described as a bioink in the present disclosure in relation to embodiments where a cell laden sol is bioprinted by extrusion printing, and which can be subsequently crosslinked to form a stable and porous scaffold or construct of the invention in any desired 3D shape, pattern or geometry.

It was not expected that a homogenously mixed/dispersed agarose: carboxymethyl chitosan: al ginate biogel could stably accommodate a homogeneous dispersion or uniformly distributed dispersion of piezoelectric nanoparticles and still be suitable for extrusion and particularly bioprinting. Piezoelectric nanoparticles are hydrophobic and have a strong tendency to aggregate in aqueous media forming clumps and aggregations of nanoparticles. To aid in dispersion in aqueous media, coating or wrapping individual nanoparticles with polymers can be used, for example, cationic polymers or polyelectrolytes to assist forming and maintaining nanoparticle dispersions in aqueous media. Conventionally, significant amounts of ultrasonication processing are required to disperse nanoparticles in aqueous media and/or to wrap or coat nanoparticle with polymers, for example, cationic polymers (e.g., PLL) or polyelectrolytes to promote dispersion. However, prior art methods for dispersing and/or coating or wrapping with polymer or polyelectrolyte are incompatible with the cell laden biogels described herein, not least for the harsh sonication treatment required. Therefore, it was an unexpected finding that the much less harsh precursor, sol or bioink preparation processes described herein would be sufficient to disperse nanoparticles in a complex biopolymer sol using only gentle steps that do not involve hours of ultrasonication at high outputs. Indeed, the inventors had previously considered that adding hydrophobic nanoparticles to a bioprintable 3D biogel without pre-coating with a dispersing agent would be detrimental to nanoparticle-cell interactions and cell survival.

### 3D Construct Preparation

Described herein is a method of forming a 3D electrogel scaffold comprising the steps of:
(i) providing an 3D electrogel precursor solution according to the second aspect;
(ii) forming the 3D electrogel precursor solution into a desired 3D shape or 3D pattern;
(iii) crosslinking the hydrogel polymers of the 3D shape or 3D pattern to form a 3D electrogel scaffold having the desired 3D shape or 3D pattern, wherein the 3D electrogel scaffold comprises a uniform dispersion of piezoelectric nanoparticles throughout a gelled and crosslinked hydrogel polymer matrix, wherein the hydrogel polymer matrix comprises a homogenous mixture of alginate, carboxymethyl-chitosan and agarose polymers.

In a related embodiment, the method comprises the additional step of: allowing a portion of the carboxymethyl-chitosan to leach out of the 3D electrogel scaffold thereby forming a porous hydrogel polymer matrix comprising interconnected pores that forms channels or pathways throughout the scaffold that support ingress, invasion and infiltration of cells, oxygen and nutrients throughout the scaffold or construct.

Suitably, the homogenous mixture is a mixture of two or more hydrogel polymers, preferably three or more hydrogel polymers. In one embodiment, the homogenous mixture of hydrogel polymer is as described above with reference to agarose, carboxymethylcellulose and chitosan polymers.

Desirably, the 3D electrogel scaffold is a crosslinked 3D electrogel scaffold. The 3D electrogel scaffold may be provided without cells as a scaffold, or with cells as a cell laden 3D electrogel scaffold from which a 3D electrogel tissue engineered construct can be derived by subjecting the cells of the cells have been subjected to culture and/or piezoelectrical stimulation. Preferably, the first aqueous solution is distilled water or a saline solution, preferably at physiological compatible pH. Suitably, the first aqueous solution is a phosphate buffered saline (PBS) solution. Preferred PBS solutions have a pH of physiological conditions, that is, about pH 7.

Desirably, a preferred 3D electrogel precursor solution is ionically conductive.

Preferred alginate, carboxymethylcellulose and agarose polymers are described in detail above.

Preferred nanoparticles are described in detail above.

Suitably, the 3D electrogel precursor solution may be adapted for 3D bioprinting. In a preferred embodiment, the 3D electrogel precursor solution may have a flow consistency index of the 3D electrogel precursor solution is from 60 to 100 Pa·s^{0.32}, more preferably, from 90 to 80 Pa·s^{0.32}, in this case the rheological properties of the prepared inks were determined using an AR-G2 rheometer (TA Instruments, USA).

In one embodiment, the 3D electrogel precursor solution is adapted for injection. When used as in vivo injectable, the gel may not be required to maintain form prior to crosslinking and so it can be less viscous than required for bioprinting, i.e., enabling a wider range of viscosity. For example, low viscosity of 10-50 cp (centipoise) is desirable for subcutaneous injection. Furthermore, in the case of injection for in situ gel formation at physiological conditions (i.e., 37° C., etc), the injectable precursor solution of the invention preferably can gel in vivo in response to ionic cross-linking and pH change. Such a gel offers specific advantages over preformed scaffolds such as: possibility of a minimally invasive implantation, an ability to fill a desired shape, and easy incorporation of various therapeutic agents such as cells. A high-viscosity, shear-thinning polymer solution or a slightly cross-linked gel may be injected through a relatively small gauge needle, with discontinuation of the injection shearing force followed by formation of a thick gel in situ. Importantly, for electrogel, in addition to alginate polymerisation when mixed with divalent cations, the chitosan component gels in response to pH changes from slightly acidic to physiological pH. Thus, particularly, at a pH lower than 6.5, a preferred chitosan suspension is injectable and at physiological pH, the polymer is believed to undergo a phase transition.

Suitably, the 3D electrogel precursor solution may be formed into a predetermined pattern or shape by one or more of extrusion, casting, printing, preferably extrusion printing which includes syringing or bioprinting. The invention also extends to a 3D electrogel scaffold obtained by the methods of the invention. In a related embodiment, the 3D electrogel scaffold comprises encapsulated cells to form a cell laden 3D electrogel scaffold. On culturing in cell culture media or in vivo in a suitably supporting cell proliferation environment, the cell laden 3D electrogel scaffold results in the formation of a 3D electrogel tissue engineered construct. On electrical stimulation by ultrasound-mediated piezoelectric stimulation (USPZ) of the nanoparticles in the construct, an advanced 3D electrogel tissue engineered construct is generated.

### 3D Electrogel Precursor Solution

In a second aspect the invention provides a three-dimensional (3D) electrogel precursor solution for forming a 3D electrogel scaffold according to the first aspect, the precursor comprising: an aqueous hydrogel polymer solution comprising a homogeneous mixture of dissolved alginate, dissolved carboxymethyl-chitosan and dissolved agarose polymers, and piezoelectric nanoparticles uniformly dispersed throughout the aqueous hydrogel polymer solution.

In one embodiment, step (i) above of providing an 3D electrogel precursor solution, comprises:
a) forming a homogenous dispersion of piezoelectric nanoparticles in a first aqueous solution;
b) completely dissolving agarose in the first aqueous solution of a homogenous dispersion of piezoelectric nanoparticles to form a second aqueous solution comprising a homogenous dispersion of nanoparticles and dissolved agarose;
c) completely dissolving carboxymethyl-chitosan in the second aqueous solution to form a third aqueous solution comprising a homogenous dispersion of nanoparticles and dissolved agarose and dissolved carboxymethyl-chitosan;
d) completely dissolving alginate in the third aqueous solution to form a fourth aqueous solution comprising a homogenous dispersion of nanoparticles and dissolved agarose, dissolved carboxymethyl-chitosan and dissolved alginate,

wherein on cooling the fourth aqueous solution forms the 3D electrogel precursor solution.

In some embodiments, the method may further comprise a step of providing cells to the 3D electrogel precursor solution in the form of a homogeneous dispersion of cells in the 3D electrogel precursor solution. Suitable cell types are discussed above.

In one embodiment, step (a) of forming a homogenous dispersion of piezoelectric nanoparticles in a first aqueous solution comprises the steps of: (i) dispersing piezoelectric nanoparticles in an aqueous solution; and optionally, (ii) disaggregating nanoparticle aggregates by sonication. While a range of different ultrasonication frequencies can be determined and employed, a frequency of 40 kHz or less is desired for disaggregation and is not damaging to the gel or nanoparticles. The sonication should be applied evenly across the solution (i.e. not focussed on one particular area) for uniform disaggregation. Suitable dispersion may be checked with bright field microscopy (i.e. submicron particle with equidistant dispersion). In one embodiment, 45 minutes of sonication in an ultrasound water bath at approximately 40 kHz may be used to disaggregate nanoparticle aggregates. Preferably, the piezoelectric nanoparticles are dispersed in the first aqueous solution in the absence of a dispersing agent such as a polyelectrolyte. Preferably, the nanoparticles are not coated with a dispersant, particularly a cationic dispersant such as PLL prior to incorporation of agarose. It is noted that agarose is an anionic polymer. Thus, suitably, step a) occurs in the absence of a dispersing agent such as polyelectrolyte. Preferably, gentle mixing, such as hand mixing, magnetic stirring and/or vortex mixing are used in favor of sonication to form and maintain a homogenous dispersion of piezoelectric nanoparticles in the polymer solutions described herein in steps b), c) and d).

Desirably, the piezoelectric nanoparticle dispersion formed in step a) may be vortexed immediately prior to use, for example, by vortexing for about one to 2 minutes vortex time prior to combination with agarose in step b). In one embodiment, step (b) involving completely dissolving agarose in the first aqueous solution comprising dispersed nanoparticles comprises: (i) heating the first aqueous solution while stirring to a temperature sufficient to melt the agarose for example, at a temperature of about 80° C., and (ii) adding the agarose to the heated first aqueous solution while stirring, preferably for around 5 to 10 minutes; (iii) heating the resultant solution in a microwave oven with agitation, preferably every 2-10 seconds, preferably around 4 seconds, until the agarose is completely dissolved; (iv) stirring the resultant solution at a temperature sufficient to melt and dissolve the polymer, (e.g., about 80° C.) (preferably for about 5 to 10 minutes) to ensure a homogenous dispersion of the nanoparticles in the third aqueous solution.

In one embodiment, step c) of completely dissolving the carboxymethyl chitosan in the second aqueous solution comprises: (i) cooling the second aqueous solution comprising a homogenous dispersion of nanoparticles and dissolved agarose to between 50° C. and 70° C., preferably about 60° C. and adding carboxymethyl chitosan; and (ii) stirring the resultant third aqueous solution at temperature sufficient to dissolve the carboxymethyl chitosan, for example, at about 60° C., preferably for about 10 mins, (ii) vortex mixing the resultant solution, preferably for about 1 to 5 minutes to ensure a homogenous dispersion of the nanoparticles in the third aqueous solution.

In one embodiment, step d) of completely dissolving alginate in the third aqueous solution comprises: (i) adding alginate to the third aqueous solution and stirring the solution to ensure a homogenous dispersion of the nanoparticles thereby forming the fourth aqueous solution, whereby on cooling to around room temperature, the fourth aqueous solution forms the 3D electrogel precursor solution. In one embodiment, the stirring in step d) may involve manually stirring for 3 to 19 minutes, preferably 5 minutes, prior to magnetic stirred for 20 to 40 minutes, preferably 30 mins with a 1 to 3 minute, preferably 1 minute vortex every 5 to 15 minutes, preferably every 10 minutes. At room temperature, the agarose begins to gel and the solution begins to thicken.

Dissolution of the polymer components is aided by heating and stirring the solutions. Dissolution is evidenced by formation of a clear solution in which all particles are dissolved. A microwave or other heating device can be used to aid dissolution. The temperature should be below boiling as this prevents significant water evaporation which can alter the polymer concentrations. Furthermore, the dissolution temperature and stirring conditions must be below those which would result in degradation of the polymers.

Desirably, the electrogel precursor solution is refrigerated at 2 to 10° C., preferably 4° C. for several hours, preferably overnight before use. Cooling to these temperatures allows the agarose to gel more completely and the solution acquires a thickness or flow consistency index which is desirable for hold a 3D pattern or 3D form for a sufficient period to allow the crosslinking step described above to take place.

Desirably, the polymers may be crosslinked by irrigating, soaking, submerging or washing the formed precursor solution, preferably the 3D electrogel precursor solution in a desired 3D pattern or 3D shape, with a suitable crosslinking solution, preferably a solution of divalent ions, such as Ca²⁺ ions, for example, in distilled water or phosphate buffered saline (PBS). PBS solutions may be preferred as they more closely resemble physiological conditions. A preferred Ca²⁺ ion solution is a CaCl₂) solution. The CaCl₂) concentration is preferably in the range of from about 10 mM to 200 mM. However, a 50 mM solution of CaCl₂) has been found to be particularly preferred in terms of resulting in a scaffold with desirable flexibility and desirable support for cell growth. Suitably, the crosslinking may process for 5, 10 or 20 minutes prior to washing to remove the crosslinking solution. For a 50 mM solution of CaCl₂) a crosslinking time of 15 minutes gives a scaffold with desirable flexibility.

Suitably, the 3D electrogel precursor solution may further comprise cells dispersed within the 3D electrogel precursor solution. Preferably, the precursor solution further comprises a dispersion of cells throughout the precursor solution. Preferably, the cells are stem cells including adult stem cells including but not limited to neural stem cells, and pluripotent stem cells. The cells have been described in detail above.

The cells may be counted to determine volume of electro-gel required to create a final cell incorporated electro-gel concentration of 2.0×10 7 cells·mL⁻¹ for the hNSC embodiment described herein. A cell suspension in +ADDs medium may be prepared and the cell suspension is incorporated into electro-gel through manual stirring for 1 minute.

### Bioprinting

A suitable precursor solution (sol) for bioprinting (also described herein as a bioink) is one which is optimally viscous for bioprinting and which comprises appropriate biomaterials to support cell viability. Furthermore, the precursor solution must be conducive to cell survival during extrusion via an extrusion head or extrusion printhead and must be of a suitable viscosity to maintain its printed shape at least for a short period until gelation occurs. As described above, a preferred 3D electrogel precursor solution may have a flow consistency index of the 3D electrogel precursor solution is 60 to 100 Pa·s^{0.32}, more preferably, about 90 to 100 Pa·s^{0.32}. Suitably where cells are present, preferably the flow consistency index of the hydrogel 3D electrogel precursor solution comprising cells is 90 to 100 Pa·s^{0.32}, which is optimised for bioprinting.

In addition, the sol must be formulated to gel by chemical crosslinking for safe cell encapsulation, structural support, and sustained cell survival for the life of the construct. Thus, a preferred bioink is one which can be crosslinked for gelation of the hydrogel-based sols post printing. A preferred bioink is supportive of cell viability whereby cells are incorporation to the sol prior to extrusion or bioprinting, or where cells are seeding to an extruded or bioprinted crosslinked scaffold after crosslinking.

When the 3-dimensional (3D) electrogel scaffold arises from 3D bioprinting, it is preferred that the 3D printing involves direct-write printing of cells for encapsulation, and in the case of stem cells, proliferation and differentiation.

Key components of a preferred bioprinting method include a 3D model of the tissue or organ to be printed, a bioprinter operating under aseptic conditions, optimally viscous bioink comprising appropriate biomaterial(s), a suitable cross-linker for gelation of hydrogel-based bioinks post-printing, and cells for incorporation to the bioink or seeding to the printed scaffold. Importantly, optimization of printing extends to the rate of printing, mechanical and chemical properties of the bioink including viscosity and modulus, and porosity of the scaffold, and cell density. For example, in some embodiments, a bioink having a viscosity ranging from about 8000 to 12000 Pa·s. is desirable. In some embodiments, a bioink having an indentation modulus ranging from about 250 Pa to 5000 Pa is desirable. In some embodiments, a bioink having a cell density of up to 80×10⁶ cells per mL is desirable.

### Forming a Scaffold

The invention further relates to a method of forming a 3D electrogel scaffold. This method comprises providing an 3D electrogel precursor solution in the form of a uniform piezoelectric nanoparticle dispersion in a solution of hydrogel polymers as described above. The 3D electrogel precursor solution may then be provided in predetermined 3D pattern or 3D form. The method then involves crosslinking the hydrogel polymers of the printed 3D electrogel precursor solution 3D pattern or 3D form to produce a crosslinked 3D electrogel scaffold.

Preferably, the step of forming the 3D electrogel precursor solution into a predetermined 3D pattern involves extrusion, for example, 3D bioprinting the 3D electrogel precursor solution into the predetermined 3D pattern or 3D shape. In some embodiments, the step of forming the 3D electrogel precursor solution into a predetermined 3D pattern or 3D form involves extruding the 3D electrogel precursor solution into the predetermined pattern for form. The pattern may be, for example, a woven, a knit, or a layered pattern.

Desirably, the crosslinking step involves washing, flooding or submerging the extruded or printed 3D electrogel precursor solution 3D pattern or form in a divalent metal ion solution as described above to induce ionic crosslinks between the hydrogel polymer strands. Suitably, the method may then further comprise washing the crosslinked electrogel construct and remove excess divalent ion solution.

In some embodiments, the step of forming the 3D electrogel precursor solution into a predetermined 3D pattern or 3D form involves directly injecting the 3D electrogel precursor solution into tissue, a cavity or a mold. The tissue or cavity or mold may be outside the body or inside the body. When the sol is provided inside the body by injection for example, the resultant extruded sol may be wash or rinsed with crosslinking agent which may be injected around the extruded sol to induce crosslinking.

### Tissue Engineering Construct

In another embodiment, the invention provides a method of forming an engineered 3D electrogel tissue construct and/or an advanced 3D electrogel tissue engineered construct. Strategies for tissue fabrication include extruding or bioprinting scaffolds which are cell laden prior to extrusion or bioprinting or which are seeded with cells following extrusion or bioprinting to result in cell laden 3D electrogel scaffold in which the cells are encapsulated within the polymers of the scaffold.

The method of forming a 3D electrogel tissue engineered construct thus comprises providing a cell laden 3D electrogel scaffold to one or more cell culture media. The method then involves culturing the cell laden 3D electrogel scaffold in a cell line specific culture media to promote cell proliferation within the scaffold thereby providing the construct. In the case of stem cells, after proliferation, the construct can be provided to a cell differentiation media and cell differentiation can be allowed to occur. After suitable periods of cell proliferation and differentiation, the method may then comprise one or more rounds of electrical stimulation via ultrasound-mediated piezoelectric stimulation (USPZ) which electrically stimulates the cells in the 3D electrogel tissue engineered construct to form an advanced 3D electrogel tissue engineered construct of the invention. Subjecting the cell laden 3D electrogel constructs to ultrasound-mediated piezoelectric stimulation (USPZ) results in promotion of cell growth and differentiation and results in the generation or development of advanced tissue features as described herein as exemplified by functional neurons and supporting neuroglia in the case of advanced 3D electrogel neural cell constructs.

It is believed that the uniformity of desirable effects observed results from the quality of the nanoparticle dispersion through the electrogel scaffold. As the nanoparticles are homogenously/evenly dispersed throughout the electrogel, the entire scaffold can be stimulated evenly/homogenously across the electrogel scaffold. The results in homogenous stimulation of cells throughout the scaffold resulting in positive effects on cell functioning including well dispersed and even maturation effects throughout the entire scaffold producing the advanced constructs of the invention.

Desirably, the cells may be selected from adult stem cells and pluripotent stem cells. Preferably, the cell line specific culture media is a cell culture medium for human pluripotent stem cells or neural stem cells. For example, a suitable medium for human pluripotent stem cells is mTeSR™1 (STEMCELL Technologies, Vancouver, BC, Canada). A suitable medium for human neural stem cell culture include Complete NeuroCult Proliferation Medium (consisting of NeuroCult NS-A Basal Medium and NeuroCult NS-A Proliferation Supplement; Human; STEMCELL Technologies). The NeuroCult NS-A Proliferation Supplement may be further supplemented with heparin (2 μg mL⁻¹; Sigma-Aldrich), epidermal growth factor (20 ng mL⁻¹; Peprotech) and basic fibroblast growth factor (20 ng mL⁻¹; Peprotech) for human neural stem cell culture, or mTeSR™1 (STEMCELL Technologies, 05850; prepared as per the manufacturer's instructions). A suitable medium for human cardiac cells is STEMdiff™ Cardiomyocyte Differentiation Medium and STEMdiff™ Cardiomyocyte Maintenance Medium (STEMCELL Technologies, Vancouver, BC, Canada). A suitable medium for expansion of mesenchymal stem cells is Gibco STEMPRO™ MSC SFM and their differentiation to bone is Gibco STEMPRO™ Osteogenesis Differentiation Kit (ThermoFisher Scientific, Carlsbad, CA, USA).

A preferred electrical stimulation step involves application of a set of ultrasound parameters that are optimized for the chosen cell and tissue type. As sonicating a system naturally produces heat due to vibration of molecules, sonicating a biological system such as cell cultures is constrained to a maximal temperature of 311.5 Kelvin, beyond which cellular damage or death can occur. In particular, as application of excessive ultrasound may result in heat transfer to the cells, the ultrasound parameters are preferably chosen to ensure excess heat is not supplied to the cells. In some embodiments, the 3D electrogel tissue engineered construct may be provided to a water bath to which controlled ultrasonication is applied. As ultrasonication increases the temperature of the water in the water bath, undesirable excessive heating can be avoided by controlling various ultrasonic parameters. Controllable ultrasound parameters include one or more of: the type of ultrasonication set up used, for example, a sonoporator probe set up or an ultrasound water bath set up. Other controllable parameters include ultrasound pulse design, such as the fundamental frequency (kHz or MHz), duty cycle (%) (the percentage of time when ultrasound signal is transmitted, or “on”), pulse repetition frequency (Hz) (frequency of periods when ultrasound is transmitted as “bursts”) and acoustic intensity (W/cm2) (pressure; how “loud” the sound wave is), and distance of the ultrasound generator from the cell laden scaffold and potential location of standing waves. Out of these variables, the biggest contributors to the ultrasound effect (especially due to heating) is the acoustic intensity and the duty cycle. After these parameters, the pulse repetition frequency and the ultrasound pulse duration and timing sequence applied are the next most significant contributors to cell modulation. In order to maximise the amount of sonication that can be applied to a biological system, each sonication system needs to be tested for temperature variation across these different parameters. As previously mentioned, increasing the acoustic intensity will have the largest effect on temperature increase as larger sound waves vibrate molecules more, following on from this reducing the duty cycle from a continuous pulse (100%) will naturally reduce the temperature increase due to sonication. To generate a piezoelectric response from the incorporated piezoelectric nanoparticles, sufficient acoustic intensity is required to deliver the necessary acoustic pressure to the nanoparticles. Previous research demonstrated acoustic intensity greater than 0.8 W/cm2 will elicit the necessary piezoelectric response from the nanoparticles to stimulate cells, however as experimental set ups vary between laboratories, the useable range of acoustic intensity may vary. This is necessary as acoustic intensity will attenuate the further it travels, as well as by the amount of different materials it needs to travel through. Finally, the interaction between all four variables can generate varying degrees of standing waves within the cell culture media. Although for 2D cultures this may not be an issue worth considering, when dealing with 3D electrogel scaffolds, the presence of standing waves can generate sufficient turbulence within the media to break the scaffolds. Standing waves can be mitigated by altering the cycle frequency and duty cycle, or alternatively by immobilising scaffolds to prevent unnecessary movement due to turbulence. In any case, electrical stimulation should not result in the 3D electrogel tissue engineered construct experiencing temperature of 38.5° C. as it this temperature and beyond, it is expected that cellular damage and/or cell death can occur. In the experiments desired herein, one set of preferred ultrasound parameters optimized for the particular setup used and for stimulation of human neural stem cells and their induction to neural tissue in particular, involves periodic application (with a Sonidel SP100 device with a planar 1 MHz/3 MHz ultrasound transducer of 25 mm diameter) of ultrasound 1 W/cm2, 50 Hz pulse repetition frequency, 50% duty cycle for 3 mins (30 seconds on/off) every hour, 5 times per day for 7 days.

For the system desired here involving neural stem cells, compared to equivalent systems without electrical stimulation, after 7 days of ultrasound stimulation based on these ultrasound parameters, cell growth and differentiation is more significant and more complex homogenously throughout the scaffold, as evidenced in terms of neuritogenesis as demonstrated by enhanced MAP2 expression, formation of many neurite extensions (both axons and dendrites), projecting over longer distances from sites of origin to synapse with other cells including neurons and neuroglia. Further, greater numbers of discrete, large, compact and uniformly distributed clusters/aggregates of cells can be observed.

Also described here are 3D electrogel scaffolds and cell laden 3D electrogel scaffolds obtained from the methods as described herein. Also described here are 3D electrogel tissue engineered construct and advanced 3D electrogel tissue engineered construct obtained from the methods as described herein.

### Medical Applications

Also described herein is a use of a 3D electrogel scaffold as described herein in one or more medical application. In another aspect, the invention provides a use of a 3D electrogel tissue engineered construct as described herein in a medical application. In another aspect, the invention provides a use of an advanced 3D electrogel tissue engineered construct as described herein in a medical application.

Desirably, the medical application may be one or more of: as a biomimetic 3D cell culture, for example, to better represent human cell growth and tissue outside the human body; as an engineered tissue; as a cell-based therapeutic, for example, to treat traumatic brain injury and neurological disorders such as epilepsy and Parkinson's disease; in vitro modelling of tissue development, cell function and dysfunction, or optimization of medical devices for in vivo tissue, such as neural tissue or cardiac tissue, interfacing.

A preferred use is one, wherein the engineered tissue is functional human tissue, including neural or cardiac functional tissue. Cardiac functional tissue includes bioengineered electrogel-based cardiac pacemaker tissue for treatment of cardiac disorders associated with native in vivo pacemaker-cell dysfunction arising from, for example, developmental and congenital defects, or acquired injury or ischemia.

The invention further provides for a use of a 3D electrogel scaffold, a cell laden 3D electrogel scaffold, a 3D electrogel scaffold, a 3D electrogel tissue engineered construct or an advanced 3D electrogel tissue engineered construct in a medical application.

Such use includes repair and/or regeneration of tissue malfunction or injury, in vitro or in vivo. For example, e.g. nerve injury, peripheral nerve injury or peripheral nerve regeneration.

Also described herein is a method of repair and/or regeneration of tissue malfunction or injury comprising the steps of:
providing a 3D electrogel scaffold, a 3D electrogel tissue engineered construct or an advanced 3D electrogel tissue engineered construct as described herein as an implant;
positioning the implant at the site of the malfunctioned tissue or injured tissue;
electrically stimulating the implant by ultrasound-mediated piezoelectric stimulation (USPZ) to promote repair and/or regeneration of tissue malfunction or injury at the implant site.

Suitably, the implant may be of a tissue type corresponding to nerve tissue, bone tissue, cardiac tissue, skin tissue, bone tissue or cartilage tissue.

In one preferred embodiment, the invention provides an electric nerve guide comprising a support and a 3D electrogel scaffold, a cell laden 3D electrogel scaffold, a 3D electrogel tissue engineered construct or an advanced 3D electrogel tissue engineered construct as described herein, disposed on said support, wherein the support is adapted to encase injured nerves. In this embodiment, ultrasound-mediated piezoelectric stimulation (USPZ) may be applied to the electric nerve guide to electrically stimulate nerves encased within or abutting the nerve guide. In a preferred electric nerve guide, the support may be a semi-permeable support for diffusion of nutrients whist acting as a barrier to scar-forming cells. Desirably, the support may be a membrane, such as a polymer membrane. Preferably, the membrane is a collagen membrane, more preferably an electrocompacted collagen membrane. Collagen electrocompaction enables collagen densification by isoelectrically compacting collagen molecules into densely packed and highly ordered bundles to form, for example. Methods for preparing electrocompacted collagen membranes are known in the art. In a preferred embodiment a cell laden 3D electrogel scaffold is disposed on the support, for example, by 3D bioprinting. Preferably, the 3D printing uses an 3D electrogel precursor solution as described herein. The support and scaffold may be cultured in cell media as described above to provide a 3D electrogel tissue engineered construct on the support. The electric nerve guide comprising a support and a 3D electrogel scaffold, a 3D electrogel tissue engineered construct or an advanced 3D electrogel tissue engineered construct may be directly implanted in the body around an injured or dysfunctional nerve.

Also described herein is the use of an electric nerve guide in the in vitro or in vivo repair and/or regeneration of tissue malfunction or injury, for example, e.g. nerve injury, peripheral nerve injury or peripheral nerve regeneration.

### Methods & Results

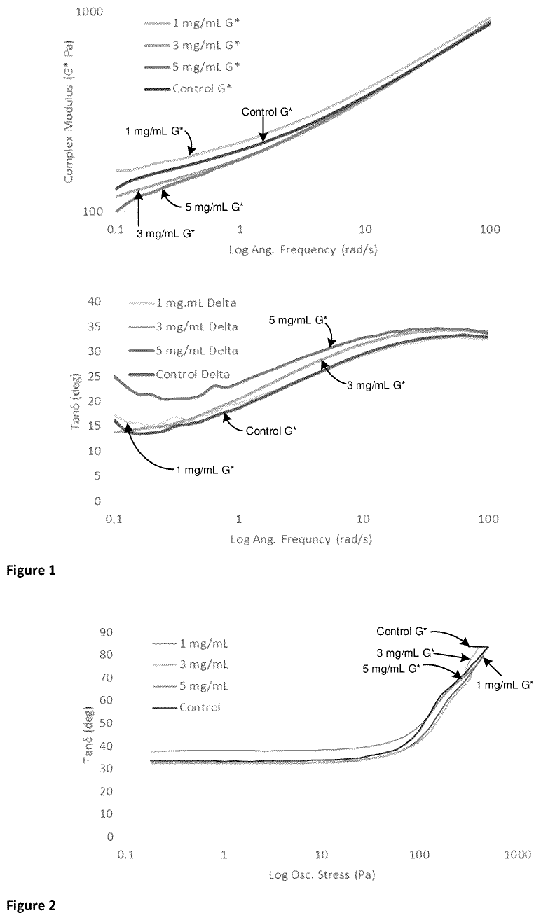

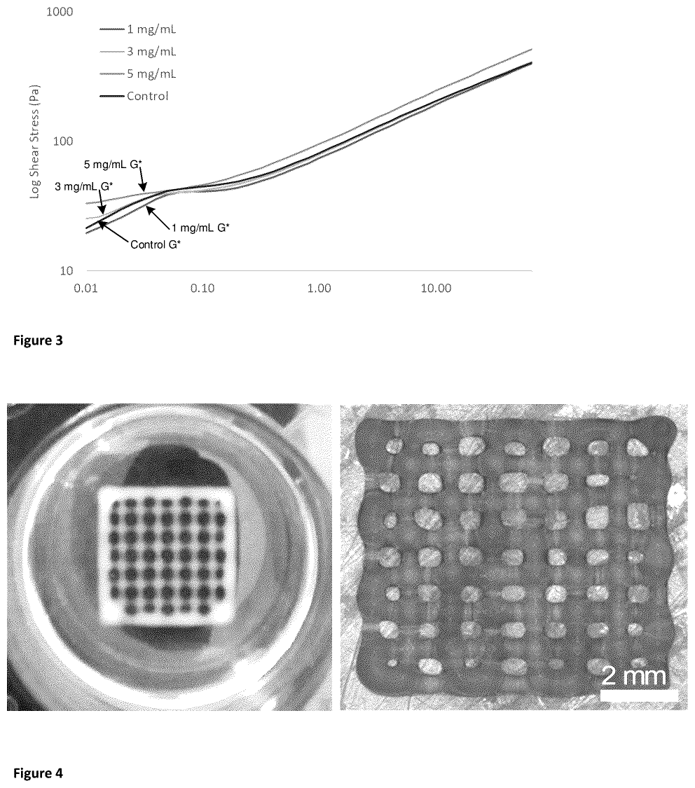

Dispersion of piezoelectric barium titanate nanoparticles—5 mg of plain piezoelectric barium titanate nanoparticles (BTNPs) with a tetragonal crystalline configuration are dispersed in 5 mL of aqueous phosphate buffer saline solution (PBS) through 1 minute of vortexing to obtain a 1 mg·mL⁻¹ dispersion. Nanoparticle aggregates are disaggregated further through 45 mins of sonication within an ultrasound water bath at approximately 40 kHz. Following sonication, the BTNP+PBS dispersion is vortexed for 1 minute prior to use.
Electro-gel preparation—Precursor sol—A 1 mg·mL⁻¹ BTNP+PBS dispersion is heated to 80° C. whilst being stirred on a magnetic stirrer, low temperature gelling agarose is added to a concentration of 1.5% w/v and stirred at 80° C. for 5 minutes. Following stirring, the agarose is completely dissolved by heating in a microwave oven, with agitation every 4 seconds. Once completely dissolved, the solution is further stirred at 80° C. for 5 minutes to ensure homogenous dispersion of BTNPs. The solution is cooled to 60° C. prior to the addition of carboxymethyl chitosan to a final concentration of 5% w/v. The solution is further stirred at 60° C. for 10 minutes followed by 2 minutes of vortexing. Once the carboxymethylchitosan has dissolved, sodium alginate is added to a final volume of 1.25% and manually stirred into the solution for 5 minutes, prior to being magnetically stirred for 30 minutes with a 1 minute vortex every 10 mins. The final solution is cooled to room temperature and refrigerated at 4° overnight before use.
Human neural stem cell incorporation—Confluent human neural stem cells (hNSCs) cultures are digested and centrifuged. Supernatant is removed and cells resuspended in 1 mL of NeuroCult NS-A Basal Medium supplemented with heparin, epidermal growth factor and basic fibroblast growth factor (+ADDS medium). A cell count is performed to determine volume of electro-gel required to create a final cell incorporated electro-gel concentration of 2.0×10 7 cells·mL⁻¹. The cell suspension is then centrifuged and supernatant removed, before resuspending cells in 50 μL of +ADDs medium. The cell suspension is incorporated into electro-gel through manual stirring for 1 minute.
Bioprinting—hNSC laden electro-gel is loaded into a 50 CC printing cartridge and centrifuged at 300 g for 1 minute to remove air bubbles. The printing cartridge is loaded into a temperature controlled (10° C.) printing magazine of an EnvisionTEC 3D-Bioplotter System. Samples are extrusion printed into a square construct (6 mm×6 mm×0.5 mm) using a 200 μm printing nozzle into a sterile 24 well plate, cooled to using a temperature controlled printing peltier cooler. The applied pressure for optimal electro-gel extrusion is 0.6 bar at a printing speed of 26 mm·sec⁻¹. Following printing, scaffolds are immersed in a 50 mM calcium chloride solution for 15 mins for cross-linking at room temperature.
Cell culture/tissue generation—Immediately after crosslinking, crosslinked scaffolds are washed by rinsing scaffolds for 1 minute three times in 37° C. DMEM/F12 medium followed by one 10 minute wash in 37° C. DMEM/F12 and one 10 minute wash in NeuroCult NS-A Basal Medium (−ADDS), before ongoing culture in +ADDS under 5% CO₂ at 37° C. Cells are allowed to proliferate under these conditions for 7 days with half volume medium changes performed ever 2-3 days. After 7 days in +ADDS medium, hNSCs laden electro-gel-based scaffolds are transferred to a 35 mm fluorodish with a glass coverslip base and differentiation is initiated by replacing medium with neural differentiation medium. The cell laden constructs are stabilised in neural differentiation medium for 2 days prior to initiating ultrasound-mediated piezoelectric stimulation (USPZ).
Ultrasound-mediated piezoelectric stimulation—Periodic USPZ stimulation is applied with a Sonidel SP100 device with a planar 1 MHz/3 MHz ultrasound transducer of 25 mm diameter. The optimal USPZ paradigm employed is as follows: 1 W·cm⁻², 50 Hz burst rate, 50% duty cycle for 3 mins (30 secs on/off) every hour, 5 times per day for 7 days. This optimised stimulation paradigm does not induce a detectable adverse increase in temperature of the cell culture medium. Fluorodishes containing the cell laden scaffolds are placed on the sonoporator probe with a polyurethane mount allowing for a 6 mm gap between the probe and fluorodish, providing room for approximately 3 mL of sterile water to be used as an acoustic transmission fluid.
Rheology of Electrogel Precursor Solution (“Sol”)—The rheological behaviour of the liquid phase (sol) electrogel precursor solution was analysed with an AR-G2 Rheometer (TA Instruments) equipped with a Peltier plate thermal controller. A 1°/40 mm cone and plate geometry was used in all measurements. The sol was allowed to equilibrate to testing temperature (10° C.) for 10 min prior to analysis. Storage modulus (G′) and loss modulus (G″) were measured as a function of frequency and strain by varying, respectively, frequency (at a constant strain 1%) and strain (at a constant frequency 10 rad/sec). Frequency sweep experiments were conducted from 0.001-100 Hz and strain sweeps were conducted from 0.1% to 1000%. Viscosity of the 3D electrogel precursor solution was investigated using a constant frequency of 10 rad/sec, a constant strain of 1% and varying the shear rate from 0.01-100/sec. A constant temperature of 10° C. for all measurements was chosen to ensure the sol maintained a gel-like structure and was equal to the temperature of 3D electrogel precursor solution when bioprinted. The impact of barium titanate nanoparticle (BTNP) concentration on the rheological behaviour of the 3D electrogel precursor solution was also investigated with BTNP concentrations ranging from 1 to 5 mg·mL⁻¹.
Frequency Sweep of 3D electrogel precursor solution—Frequency sweep measurements of the 3D electrogel precursor solution with varying concentrations of BTNPs were compared with control gel sol (without BTNPs). Control gel sol demonstrated shear thinning behaviour with increasing frequency as evidenced by a linear increase in complex modulus (G*; FIG. 1). The increase in tan δ approaching unity before plateauing suggests the gel sol retains its gel-like behaviour (tan δ<45°), even at high frequencies at constant strain of 1% (FIG. 1). Surprisingly, addition of BTNPs to the gel sol (to form electrogel) did not have a significant effect on frequency behaviour, for either complex modulus or tan δ measurements.
Strain Sweep of 3D electrogel precursor solution—Strain sweep measurements indicated a greater effect of strain on the 3D electrogel precursor solution rheological behaviour than frequency. The 3D electrogel precursor solution demonstrated liquid-like behaviour at high strain as evidenced by the yield point at ˜100 Pa. The yield point of the 3D electrogel precursor solution was relatively consistent across all BTNP concentrations and similar to control gel sol (FIG. 2).
Flow Curve of 3D electrogel precursor solution—As seen from the frequency and strain sweeps, the 3D electrogel precursor solution undergoes shear thinning, with viscosity decreasing with increasing shear rate. Following a Power Law model (level of fit, R 2), the flow behaviour and flow consistency indices can be compared between 3D electrogel precursor solutions with different concentrations of BTNPs (FIG. 3, Table 1). There was negligible change in the flow behaviour index (0.33-0.34) with addition of BTNPs (Table 1), supporting stable shear thinning characteristics of the 3D electrogel precursor solution regardless of BTNPs. Increasing BTNP concentration, however, resulted in an increase in the flow consistency index, suggesting that BTNPs contribute to thickening of the 3D electrogel precursor solution. However, a 1 mg·mL⁻¹ BTNP concentration resulted in a reduction of the consistency below that of control 5 gel sol (Table 1).

TABLE 1
Flow consistency index and behaviour index values for the
shear thinning region of the electrogel with varying amounts
of BTNPs compared to control gel (without BTNPs).

| | Flow Consistency | | |
|---|---|---|---|
| | Index | Flow | Behaviour |
| Condition | (Pa · sⁿ) | Index | R² |
| Control | 94.44 | 0.33 | 0.987 |
| 1 mg/mL | 87.34 | 0.33 | 0.984 |
| 3 mg/mL | 93.81 | 0.34 | 0.98 |
| 5 mg/mL | 111.44 | 0.33 | 0.978 |

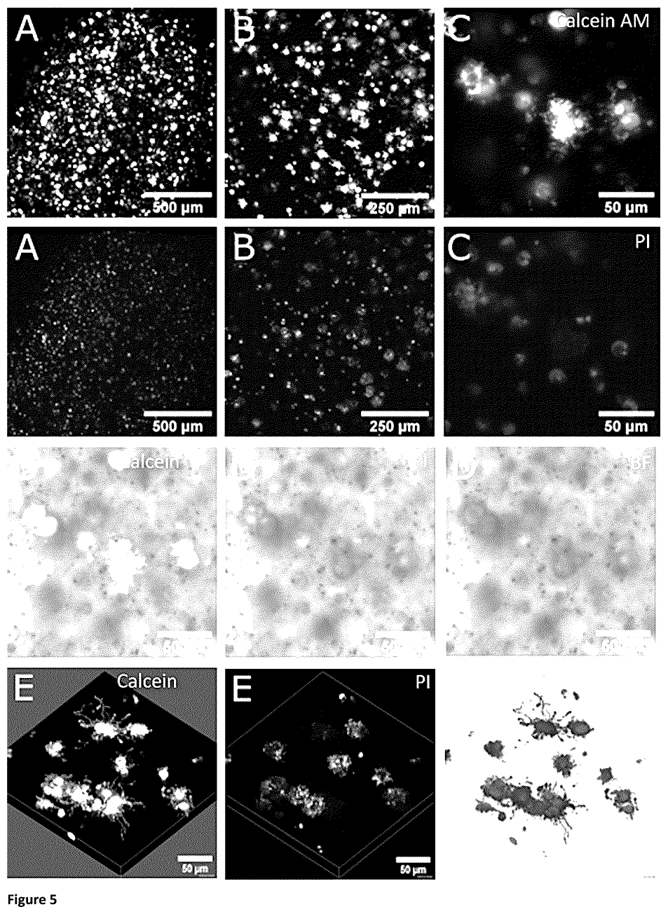

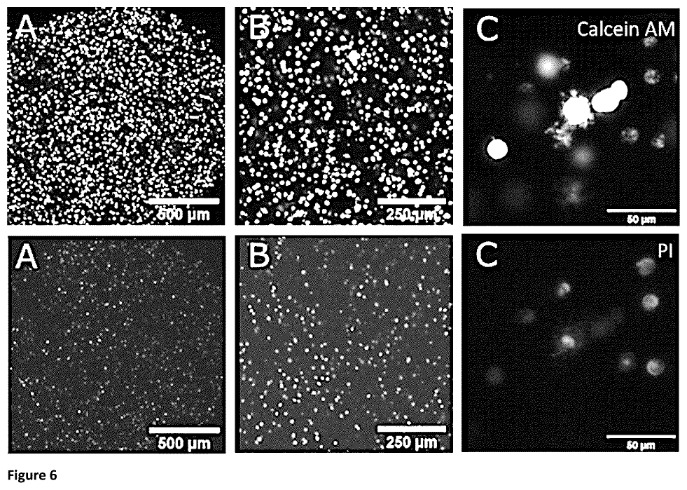

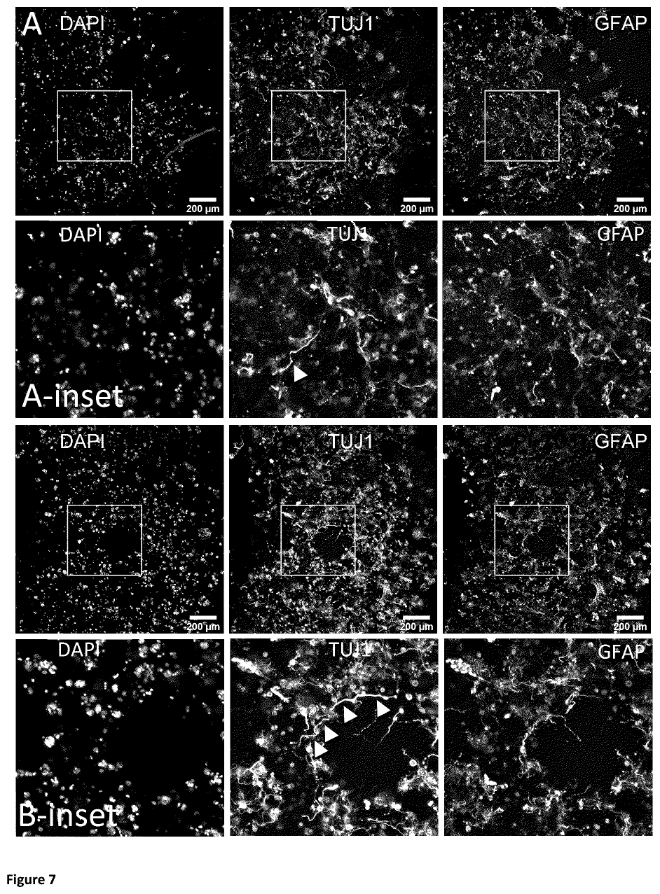

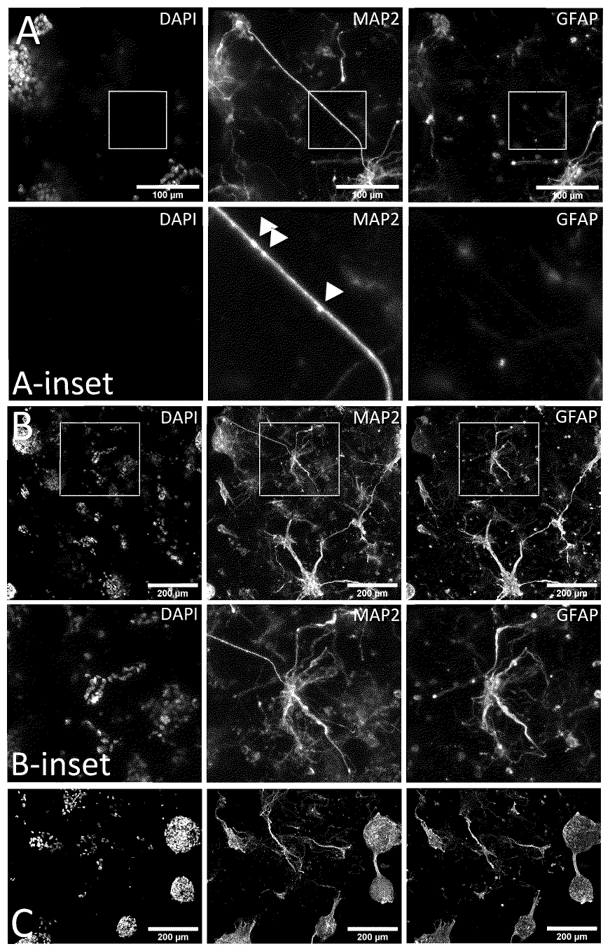

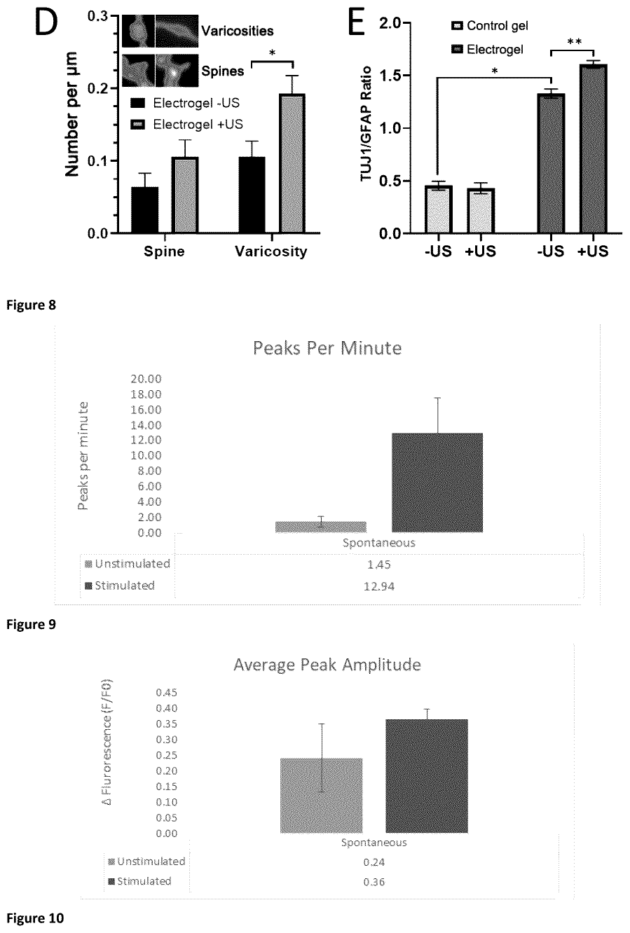

Printability of 3D electrogel precursor solution—The exemplary 3D electrogel precursor solution described herein can be direct-write printed and rapidly undergoes gelation by chemical (divalent cationic) cross-linking to form a porous three-dimensional (3D) scaffold construct with or without encapsulated cells (FIG. 4).
Demonstration of Human Neural Stem Cell Viability Within Printed Electrogel—Live (Calcein AM) and dead (propidium iodide) cell staining of human neural stem cells (hNSCs) demonstrated cell survival (viability) within electrogel 7 days after 3D printing. More specifically, following printing and gelation by ionic-crosslinking of hNSC-laden 3D electrogel precursor solution, cell viability was similar to that of printed control gel (without BTNPs) (FIGS. 5 and 6). Moreover, hNSCs within electrogel occurred as single cells and aggregates of cells (the latter indicative of stem cell proliferation), and developed many filopodia extensions into the electrogel (FIG. 5E, F). In contrast, hNSCs within control gel maintained a rounded morphology with fewer cell aggregates apparent and minimal neurite extensions into the gel).
Immunophenotyping Of Differentiated Human Neural Stem Cells Within Printed Electrogel—Immunophenotyping of hNSC-laden electrogel scaffolds or control gel (without BTNPs) scaffolds was performed 14 days after printing (7 days hNSC proliferation, 7 days cell differentiation). Printed scaffolds were fixed and immunocytochemistry was performed for early neuronal specific marker Tubulin (TUJ1) and glial cell marker glial fibrillary acid protein (GFAP) (FIG. 7). Qualitative analysis indicated electrogel scaffolds were characterised by higher labelling of TUJ1 expressing cells, with greater neuritogenesis supported by greater neurite number and longer neurites (FIG. 7B). Control gel scaffolds comprised higher numbers of GFAP expressing cells, compared to TUJ1 expressing cells (FIG. 7A).
Immunophenotyping Of Differentiated Human Neural Stem Cells Within Printed Electrogel Following Ultrasound-Mediated Piezoelectric Stimulation—Immunophenotyping of hNSC-laden electrogel scaffolds was performed following USPZ stimulation for 7 days. Printed hNSC-laden electrogel scaffolds were first cultured in neural differentiation medium 3 days prior to initiating stimulation, resulting in a total of 10 days differentiation. Compared to unstimulated (no ultrasound exposure) constructs, USPZ stimulation resulted in more significant and complex neuritogenesis, evident as many neurite extensions (dendrites and axons), projecting over longer distances from their sites of origin to synapse with other cells, including neurons and associated neuroglia (˜200 μm in length, FIG. 8A). Stimulated constructs also exhibited greater numbers of discrete, large, compact and uniformly distributed clusters/aggregates of neural cells (FIG. 8B, C) with more neurites and bundles of neurites radiating per cluster (FIG. 8B-inset). Quantitative analyses confirmed increased numbers of synaptic varicosities/boutons (swellings) formed along the length of individual neurites following stimulation (FIG. 8D). Varicosities were defined as having a low aspect ratio of length to diameter (<1). Although not statistically significant, a trend towards increased numbers of spines (protrusions) along stimulated neurites was also measured (FIG. 8D). Spines were defined as having high aspect ratio of length to diameter (>1). Finally, quantitative analysis of Tuj1 and GFAP labelling revealed increased neuronal relative to glial cell induction respectively within electrogel compared to control gel (without BTNPs), which was further enhanced by USPZ stimulation (FIG. 8E). The observed effects of USPZ stimulation demonstrates electrical activation of electrogel resulting in augmented neural cell growth, neuronal induction and neuritogenesis with enhanced synaptogenesis, neural networking and tissue formation.
Live-Cell Calcium Imaging of Differentiated Human Neural Stem Cells Within Printed Electrogel Following Ultrasound-Mediated Piezoelectric Stimulation—Live-cell calcium imaging was used to investigate the activity of differentiated hNSCs within printed electrogel constructs following ultrasound-mediated piezoelectric (USPZ) stimulation for 7 days. Again, printed hNSC-laden electrogel scaffolds were first cultured in neural differentiation medium 3 days prior to initiating stimulation, resulting in a total of 10 days differentiation followed by spontaneous calcium flux imaging using fluorescent calcium indicator Fluo-4 Am (Life Technologies). For each sample assessed, 80 cells were selected and defined as a region of interest (ROI). Fluorescence intensity (mean grey value, F) per ROI over the time series was corrected for background intensity (F0) at each corresponding time point. Statistical analyses of data were performed in IBM SPSS Statistics for Windows 2012 (Version 21.0). An F-test was initially performed to test the assumption of homogeneity of variance (P>0.05). A student t-test was then conducted to compare USPZ stimulated and unstimulated (without ultrasound exposure) samples. USPZ stimulated cells within electrogel exhibited elevated calcium flux with an increased spontaneous firing rate (peaks per minute; FIG. 9) as well as spike amplitude (average peak amplitude; FIG. 10) compared to unstimulated (ie. without ultrasound exposure) cells within electrogel. The increase in firing rate and spike amplitude of cells in USPZ stimulated electrogel is consistent with electrical activation of electrogel resulting in an effect on neural cell and neuronal tissue function.
e-Nerve Guide

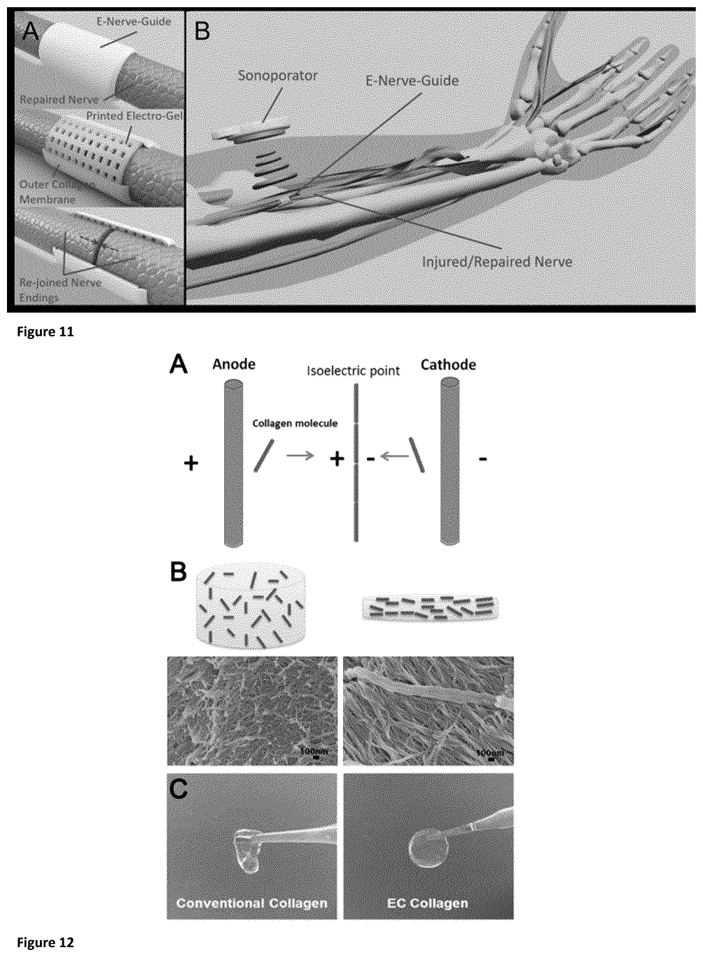

The e-nerve guide of the present invention provides a solution to treating and/or augment peripheral nerve injury (PNI) following injury or restoration of function to dysfunctional nerve tissue. The e-nerve-guide is designed to be wrapped around and rejoin severed or severely damaged peripheral nerves, while concomitantly wirelessly-electrically-stimulating axonal growth for active nerve regeneration and repair as e-stimulation has of a damaged nerve, encourages axonal regeneration and function for nerve repair. The electric nerve-guide (e-nerve-guide) of the invention enables multimodal nerve repair. The e-nerve-guide comprises an outer protective type I collagen membrane and an inner 3D printed electro-gel coating (FIG. 11). The device can be created in less than 24 h, is resorbable, flexible (to wrap around injured nerves), semi-permeable (to allow diffusion of nutrients whist acting as a barrier to scar-forming cells), and uniquely enables wireless ultrasound-mediated electrical-stimulation (e-stim) of encased nerves. The device may be used in vitro and in vivo for the ability to augment peripheral nerve regeneration following injury.

Similar to conventional nerve-guides, the e-nerve-guide allows tensionless repair of a nerve, and seals the repair-site to prevent invasion by unwanted cells (i.e., fibrosis) while allowing nutrient diffusion. Remarkably, the outer membrane of our nerve-guide is made entirely of type I bovine collagen for a defined, hypo-immunogenic and simpler device for manufacturing and regulatory approval (FIG. 12). Conventionally prepared collagen membranes lack the mechanical properties for required structural integrity thereby necessitating mixing with synthetic or other mechanically superior materials. The present inventors have efficiently and rapidly produced mechanically robust membranes (more akin to natural collagen-based structures/tissues; e.g. corneal tissue) from pure collagen by novel electrocompaction method.

The membranes can be prepared to match the dimensions of a nerve, are easily handled by a surgeon, and able to be secured around a nerve using a running suture. In summary, by integrating the collagen membranes with the electro-gel of the invention, results in a unique nerve-guide that acts as both a protective conduit and electroceutical device to directly augment nerve regeneration and restoration of function by stimulating and channelling axonal growth.

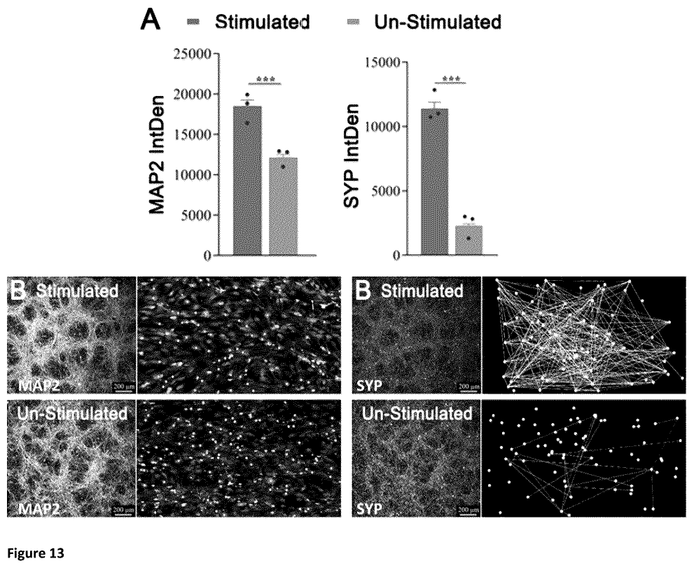

The electrogel is 3D printed on the inner surface of the collagen membrane so as to contact and stimulate the damaged nerve when encased by wrapping. Therefore, the electro-gel enables a surgeon or other clinician to perform targeted stimulation to treat the damaged area of the nerve, and simultaneously augment traditional nerve-guide function for nerve regeneration. The stimulation platform has been extensively tested for electrifying human neural stem cells in 3D culture to augment development to functionally interconnected nerve cells forming 3D neural tissue (FIG. 13).

### Discussion

hNSCs can be induced to functional neurons and supporting neuroglia, with gene expression analysis by RT-qPCR indicating differentiation of stem cells in the 3D constructs may be advantageous compared to conventional 2D platforms for accelerated neuronal, neuroglial, and synapse formation. Interestingly, the highly expressed glial marker GFAP is consistent with its key role in central nervous system (CNS) processes including astrocyte—neuron interactions as well as cell—cell communication, with the latter extending to astrocyte mediated synapse formation and function. The system may also bias neuronal differentiation to GABAergic lineage, making it attractive for inhibitory neuronal and tissue modelling. Notwithstanding, the occurrence of other neuronal subtypes including glutamatergic and serotonergic, indicate the potential for more expansive modelling, with the possibility of enriching subtype neuronal expression through, for example, cytokine supplementation.

Finally, calcium imaging of functioning neurons within the 3D construct together with SEM imaging of neurons and neurites with complex 3D morphologies demonstrate platform utility for modelling human neural cell form and activity and fabricating functional 3D human neural tissue. As such, the platform is amenable to translational drug-screening in vitro, studying human neurodevelopment and disease, and possibly neural tissue engineering for CNS tissue replacement.

1. A 3-dimensional (3D) electrogel scaffold comprising piezoelectric nanoparticles uniformly dispersed throughout a homogenous hydrogel polymer matrix, wherein the hydrogel polymer matrix is gelled and comprises crosslinked alginate, carboxymethyl-chitosan and agarose polymers.

2. A 3D scaffold according to claim 1, wherein the homogenous hydrogel polymer matrix is a porous hydrogel polymer matrix.

3. A 3D scaffold according to claim 1, wherein individual piezoelectric nanoparticles or agglomerates of nanoparticles are uncoated that is they are free of a distinct layer or coating of dispersant and/or cationic polymer.

4. (canceled)

5. A 3D scaffold according to claim 1, wherein the piezoelectric nanoparticles are dispersed within the matrix with the aid of a polymer coating around the nanoparticles that includes one or more of agarose, poly-D-lysine, poly-ornithine, gum arabic.

6. A 3D scaffold according to claim 2, wherein the porous structure has a spongy or trabecular porous structure.

7. (canceled)

8. A 3D scaffold according to claim 6, comprising a porous hydrogel polymer matrix wherein a porous structure has a combination of large and small pores sized from 25-50 pm and 10-20 pm respectively.

9. A 3D scaffold according to claim 1, wherein the average diameter of individual nanoparticles in the electrogel is 500 nm or less.

10. A 3D scaffold according to claim 1, wherein the average diameter of nanoparticle agglomerations in the matrix is from 400 nm to 200 μm, preferably <1500 nm.

11. A 3D scaffold according to claim 1, further comprises a uniform dispersion of cells throughout the porous hydrogel polymer matrix.

12. (canceled)

13. A 3D scaffold according to claim 11, wherein the cells are one or more types of stem cells.

14. A 3D scaffold according to claim 1, wherein the hydrogel matrix comprises alginate, carboxymethyl-chitosan and agarose polymers in a ratio of 0.5-5%:5%:1.5% (w/v).

15-16. (canceled)

17. A 3D scaffold according to claim 1, wherein the piezoelectric nanoparticles are present at a concentration of up to 7.5 mg/ml, preferably of up to 5 mg/ml.

18. A 3D scaffold according to claim 1, wherein the hydrogel is electronically conductive.

19. A 3D scaffold according to claim 1, wherein the piezoelectric nanoparticles are in the form of nanospheres, nanofibers, nanotubes, nanocubes, or combinations thereof.

20. A 3D scaffold according to claim 1, wherein the piezoelectric nanoparticles are selected from the group consisting of: barium titanate nanoparticles (BTNPs), boron-nitride nanoparticles, poly(vinylidene fluoride) (PVDF) nanoparticles and combinations thereof.

21-49. (canceled)

50. A 3D scaffold according to claim 13, in the form an engineered scaffold or tissue which is functional human tissue, including neural, bone or cardiac functional tissue.

51-55. (canceled)

56. A 3D scaffold according to claim 13, in the form of an electric nerve guide comprising a support and the 3D electrogel scaffold disposed on an inner surface of said support, wherein the inner surface of the support encases injured nerves.

57. A 3D scaffold according to claim 56, wherein the support is a semi-permeable support for diffusion of nutrients whist acting as a barrier to scar-forming cells or wherein the support is a membrane comprising a polymer membrane.

58. (canceled)

59. A 3D scaffold according to claim 57, wherein the membrane is an electrocompacted collagen membrane.

60-62. (canceled)

63. A method of repair and/or regeneration of tissue malfunction or injury comprising the steps of:
providing a 3D electrogel scaffold, a 3D electrogel tissue engineered construct or an advanced 3D electrogel tissue engineered construct as an implant;
positioning the implant at the site of the malfunctioned tissue or injured tissue;
electrically stimulating the implant by ultrasound-mediated piezoelectric stimulation (USPZ) to promote repair and/or regeneration of tissue malfunction or injury at the implant site.

64-66. (canceled)
